SEQUENCE LISTING (18 sequences)

SEQ ID NO: 1 - protein, 6 aa, Artificial Sequence
  peptide siderophore
  VARIANT (1)..(3): Xaa is any unknown, potentially variable or modified amino acid
  VARIANT (4)..(4): Xaa is a modified amino acid comprising a side chain with an N-nitroso-hydroxylamine ligand
  VARIANT (5)..(5): Xaa is any unknown, potentially variable or modified amino acid
  VARIANT (6)..(6): Xaa is a modified amino acid comprising a side chain with an N-nitroso-hydroxylamine ligand
XXXXXX

SEQ ID NO: 2 - protein, 6 aa, Artificial Sequence
  peptide siderophore
  VARIANT (4)..(4): Xaa is a modified amino acid comprising a side chain with an N-nitroso-hydroxylamine ligand
  VARIANT (6)..(6): Xaa is a modified amino acid comprising a side chain with an N-nitroso-hydroxylamine ligand
DTTXGX

SEQ ID NO: 3 - protein, 5 aa, Artificial Sequence
  peptide siderophore
  VARIANT (1)..(2): Xaa is any unknown, potentially variable or modified amino acid
  VARIANT (3)..(3): Xaa is a modified amino acid comprising a side chain with an N-nitroso-hydroxylamine ligand
  VARIANT (4)..(4): Xaa is any unknown, potentially variable or modified amino acid
  VARIANT (5)..(5): Xaa is a modified amino acid comprising a side chain with an N-nitroso-hydroxylamine ligand
XXXXX

SEQ ID NO: 4 - protein, 10 aa, Artificial Sequence
  predicted sequence
DMITFGCLFK

SEQ ID NO: 5 - protein, 10 aa, Artificial Sequence
  predicted sequence
DLTKVGHVGK

SEQ ID NO: 6 - protein, 10 aa, Artificial Sequence
  predicted sequence
DFWLIGMVHK

SEQ ID NO: 7 - protein, 10 aa, Artificial Sequence
  predicted sequence
DFWNIGMVHK

SEQ ID NO: 8 - protein, 10 aa, Artificial Sequence
  predicted sequence
DVHRTGLVAK

SEQ ID NO: 9 - protein, 10 aa, Artificial Sequence
  predicted sequence
DILMIGLIWK

SEQ ID NO: 10 - protein, 10 aa, Artificial Sequence
  predicted sequence
DVHRTGLVAK

SEQ ID NO: 11 - DNA, 20 bp, Artificial Sequence
  primer
gcaggctgaggattgcggcg

SEQ ID NO: 12 - DNA, 31 bp, Artificial Sequence
  primer
ggtttaattaacgcccgttcgataaagccgc

SEQ ID NO: 13 - DNA, 29 bp, Artificial Sequence
  primer
ggtggtacctcgcgtttctcggccgtctc

SEQ ID NO: 14 - DNA, 29 bp, Artificial Sequence
  primer
ggtgctagctcgtgtcgaggttcagcgcg

SEQ ID NO: 15 - DNA, 20 bp, Artificial Sequence
  primer
catcggcggcgcgagtgtcg

SEQ ID NO: 16 - DNA, 20 bp, Artificial Sequence
  primer
cctcgctatccgtgtgcgcg

SEQ ID NO: 17 - DNA, 29 bp, Artificial Sequence
  primer
ggtggtacccccgtcagtagctgaacagg

SEQ ID NO: 18 - DNA, 31 bp, Artificial Sequence
  primer
ggtttaattaaaacgaccctgccctgaaccg

(19) United States
(12) Patent Application Publication
(10) Pub. No.: US 2021/0054028 A1
(43) Pub. Date: Feb. 25, 2021

(54) BACTERIAL SIDEROPHORE GRAMIBACTIN

(71) Applicant: Leibniz-Institut für Naturstoff-Forschung und Infektionsbiologi - Hans-Knöll-Institut, Jena (DE)

(72) Inventors: Christian Hertweck, Leipzig (DE); Keishi Ishida, Jena (DE); Ron Hermenau, Jena (DE)

(21) Appl. No.: 16/772,731

(22) PCT Filed: Dec. 14, 2018
(86) PCT No.: PCT/EP2018/084952

§ 371 Date: Jun. 12, 2020

(30) Foreign Application Priority Data
Dec. 15, 2017 (EP) 17207667.1

(51) Int. Cl. C07K 11/02; C05F 11/08; C05D 9/02

(52) U.S. Cl. CPC C07K 11/02; C05D 9/02; C05F 11/08

(57) ABSTRACT

A peptide siderophore, includes one or more N-nitroso-hydroxylamine ligands. The peptide siderophore can include two side chains, each with a N-nitroso-hydroxylamine ligand, and a further side chain with one or more hydroxy carboxylic acid, hydroxamate, catecholate and/or salicylate ligands. The siderophore can be cyclic. A bacterial cell can express the peptide siderophore, and a composition can include the peptide siderophore, for example in the form of its corresponding iron-complex or bacteria. A method for promoting plant growth, promoting root growth or development, improving stress tolerance in a plant, increasing crop yields of a plant, delivering nitric oxide (NO) to a plant, and/or enhancing chlorophyll production in a plant includes administering the peptide siderophore, bacteria expressing the peptide siderophore or a composition including the peptide siderophore to the plant.

### FIELD

The invention relates to a peptide siderophore, comprising one or more N-nitroso-hydroxylamine ligands. In a preferred embodiment, the peptide siderophore comprises two side chains, each with a N-nitroso-hydroxylamine ligand, and a further side chain, which may have a N-nitroso-hydroxylamine ligand, or one or more hydroxy carboxylic acid, hydroxamate, catecholate and/or salicylate ligands, wherein the siderophore is preferably cyclic. In another preferred embodiment, the peptide siderophore comprises two side chains, each with a N-nitroso-hydroxylamine ligand, and a further side chain with one or more hydroxy carboxylic acid, hydroxamate, catecholate and/or salicylate ligands.

The invention further relates to a bacterial cell producing and/or secreting a peptide siderophore of the invention. The invention further relates to a composition comprising a peptide siderophore of the invention, for example in the form of its corresponding iron-complex, or comprising bacteria of the invention.

The invention further relates to a method for promoting plant growth, promoting root growth or development, improving stress tolerance in a plant and/or for increasing crop yields of a plant, and/or for delivering nitric oxide (NO) to a plant and/or for enhancing chlorophyll production in a plant by administering the peptide siderophore, bacteria producing and/or secreting the peptide siderophore or a composition comprising the peptide siderophore to said plant.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 33505614_1.txt, the date of creation of the ASCII text file is Sep. 15, 2020, and the size of the ASCII text file is 5.02 KB.

### BACKGROUND OF THE INVENTION

Life on earth critically depends on iron (Fe), which plays an eminent role in a broad range of biochemical reactions including energy production, biosynthesis and replication. Although iron is one of the most abundant elements in Earth's crust, it is not bioavailable under aerobic conditions. To capture this important element from the environment, sophisticated molecular strategies using specific iron transporters (siderophores) have evolved. Microorganisms and plants can cope with the limited supply of soluble iron by sequestering multidentate iron(III)-binding agents named siderophores (1).

In light of the hundreds of known siderophore structures it is nonetheless remarkable that there is only little variation in the types of donor sites. Almost exclusively, α-hydroxy-carboxylic acids, hydroxamates, catecholates, and salicylates (FIG. 1A) are used as ligands to capture iron from the environment (1). Whereas these highly Fe-affine ligands provide an advantage in pathogenic interactions (1), iron mobilization needs to be fine-tuned among mutualists sharing ecosystems such as the rhizosphere, a complex matrix of plant roots, microbes and inorganic matter (2).

In plants iron is essential for chlorophyll production and thus correlates with plant growth and high crop yields. Root-associated bacteria may serve the host plant in providing solubilized iron, thus supporting growth and fitness in return for nutrients provided through root exudates (3).

Despite advances in understanding the mechanisms of Fe sequestering by plants, a need exists in the field of agricultural sciences for novel means that effectively provide soluble iron to plants in order to improve plant growth, stress tolerance and/or crop yields.

### SUMMARY OF THE INVENTION

In light of the prior art the technical problem underlying the present invention is to provide alternative or improved means for the provision of iron to plants to improve plant growth, stress tolerance and/or crop yields. A further objective of the invention may be considered the provision of means for the provision of nitric oxide (NO) to plants and/or for the improved production of chlorophyll in plants.

This problem is solved by the features of the independent claims. Preferred embodiments of the present invention are provided by the dependent claims.

The invention relates to an isolated peptide siderophore, comprising one or more N-nitroso-hydroxylamine ligands.

Microorganisms and plants capture life-essential iron from the environment by sequestering iron(III)-binding agents named siderophores. The inventors found that rhizosphere bacteria (Burkholderia graminis) produce a unique siderophore (gramibactin) with a dual function: its unusual N-nitroso-hydroxylamine (diazeniumdiolate) ligands efficiently bind iron and serve as nitric oxide (NO) donors.

PET-CT tracer studies and supplementation experiments revealed that maize plants take up iron from the complex, which results in a marked increase (by 50%) in chlorophyll production. In vitro assays and in vivo fluorescence imaging showed that gramibactin liberates NO, a pluripotent plant hormone mediating iron homeostasis. The findings of the invention have broader ecological and agricultural implications, since gramibactin biosynthesis genes are conserved in numerous plant-associated bacteria, including species associated with rice, maize and wheat, the top three crops worldwide.

It should be highlighted that chelating properties of N-nitroso-hydroxylamines, which are isosteric to hydroxamates, have only been previously reported for synthetic compounds, such as the copper-complexing reagent cupferron (10). This functional group has not been previously described for iron-chelating compounds or for naturally occurring iron chelators isolated from soil bacteria.

In a preferred embodiment, the peptide siderophore comprises two side chains, each with a N-nitroso-hydroxylamine ligand, and a further side chain, which may have a N-nitroso-hydroxylamine ligand, or one or more hydroxy carboxylic acid, hydroxamate, catecholate and/or salicylate ligands, wherein the siderophore is preferably cyclic.

In a preferred embodiment, the peptide siderophore of the invention comprises two side chains, each with a N-nitroso-hydroxylamine ligand, and a further side chain with one or more hydroxy carboxylic acid, hydroxamate, catecholate and/or salicylate ligands, preferably a hydroxy carboxylic acid ligand.

In a preferred embodiment, the peptide siderophore of the invention comprises a peptide of X1-X2-X3-Gra1-X4-Gra2 (SEQ ID NO: 1), wherein:
a. X1 to X4 are amino acids and wherein at least one of X1 to X4 comprises a side chain with at least two atoms capable of forming one or more Fe-chelating ligands, preferably a side chain with N-nitroso-hydroxylamine, hydroxy carboxylic acid, hydroxamate, catecholate, or salicylate ligands; and
b. Gra1 and Gra2 may be the same or different and are amino acids comprising a side chain with an N-nitroso-hydroxylamine ligand.

In a preferred embodiment, the peptide siderophore of the invention comprises a peptide of Asp-Thr1-Thr2-Gra1-Gly-Gra2 (SEQ ID NO: 2), wherein Asp comprises a side chain with an additional hydroxy group adjacent to the carboxylic acid group.

In a preferred embodiment, the peptide siderophore of the invention is characterized in that the peptide is cyclic, preferably with a cyclic structure comprising 2 to 10 amino acids, preferably 3 to 6 amino acids.

In a preferred embodiment, the peptide siderophore of the invention is characterized in that the side chain of Thr1 and the C-terminus of Gra2 are linked to form a cyclic peptide.

In a preferred embodiment, the peptide siderophore of the invention comprises a peptide of X2-X3-Gra1-X4-Gra2 (SEQ ID NO: 3), wherein:
a. X2 to X4 are amino acids and wherein at least one of X2 and/or X3 comprises a side chain with at least two atoms capable of forming one or more Fe-chelating ligands, preferably a side chain with N-nitroso-hydroxylamine, hydroxy carboxylic acid, hydroxamate, catecholate, or salicylate ligands; and
b. the side chain of X2 and the C-terminus of Gra2, or the N-terminus of X2 and the C-terminus of Gra2, are linked to form a cyclic peptide.

In a preferred embodiment, the peptide siderophore of the invention is characterized in that the N-nitroso-hydroxylamine ligand, such as in Gra1 and/or Gra2, is present in an amino acid of the structure:

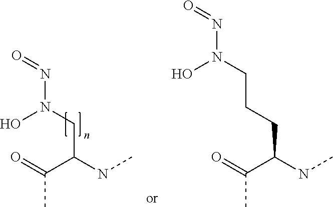

wherein n is a value from 1 to 7, preferably 2-5. The dotted line may represent a bond to a neighboring atom, for example in the context of an amino acid or a peptide chain.

A further aspect of the invention relates to a cyclic peptide siderophore as described herein, according to Formula I:

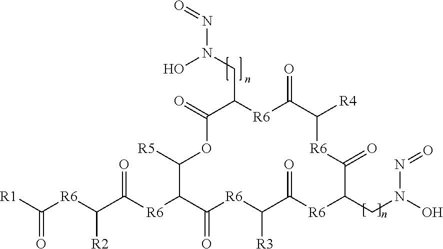

wherein:
R1 is C1-C12 alkyl, alkoxy, carboxyl or hydroxy carboxyl, or one or more amino acids (or the remaining part of an amino acid (—NH—CR—) additional to the C═O group to which R1 is attached, wherein R is the amino acid side chain);
R2 to R5 may be the same or different, and are selected from the group consisting of a side chain of an amino acid, H, OH, C1-C7 alkyl, alkoxy, carboxyl or hydroxy carboxyl;
R6 can be the same or different, wherein R6 is O or NH, and wherein at least one of R6 is NH;
n is a value from 1 to 7, preferably 2 to 5, wherein n for different substituents may be the same or different;
and wherein R1 to R5 comprise between them at least two atoms capable of forming one or more Fe-chelating ligands.

A further aspect of the invention relates to a cyclic peptide siderophore as described herein, according to Formula II:

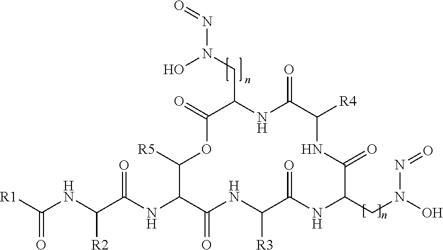

wherein:
R1 is C1-C12 alkyl, alkoxy, carboxyl or hydroxy carboxyl, or one or more amino acids (or the remaining part of an amino acid (—NH—CR—) additional to the C═O group to which R1 is attached, wherein R is the amino acid side chain);
R2 to R5 may be the same or different, and are selected from the group consisting of a side chain of an amino acid, H, OH, C1-C7 alkyl, alkoxy, carboxyl or hydroxy carboxyl;
n is a value from 1 to 7, preferably 2 to 5, wherein n for different substituents may be the same or different;
and wherein R1 to R5 comprise between them at least two atoms capable of forming one or more Fe-chelating ligands.

In a preferred embodiment of Formula I or II, the cyclic peptide siderophore is characterized in that:
R1 is C1-C12 alkyl, alkoxy, carboxyl or hydroxy carboxyl;
R2 is the side chain of Asp or Glu, optionally comprising an additional hydroxy group adjacent (alpha or beta) to the carboxylic acid group;
R3 is the side chain of Ser, Thr, Asn or Gln;
R4 is H or C1-C7 alkyl;
R5 is H or C1-C7 alkyl;
n is a value from 2 to 5.

In some embodiments, R1 of Formula I or II is H, OH, C1-C12 alkyl, alkoxy, carboxyl or hydroxy carboxyl, or one or more amino acids (or the remaining part of an amino acid (—NH—CR—) additional to the C═O group to which R1 is attached, wherein R is the amino acid side chain)

The cyclic peptide siderophore in a preferred embodiment exhibits the following structure:

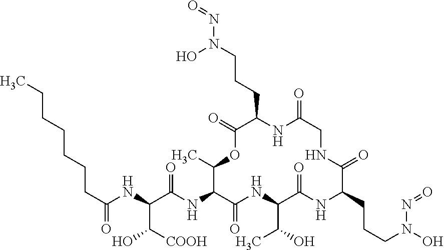 [SMILES: CCCCCCCC(=O)N[C@@H](C(=O)N[C@@H]1C(=O)N[C@H]([C@@H](C)O)C(=O)N[C@H](CCCN(O)N=O)C(=O)NCC(=O)N[C@H](CCCN(O)N=O)C(=O)O[C@@H]1C)[C@@H](O)C(=O)O]

A further aspect of the invention relates to a bacterial cell producing and/or secreting a peptide siderophore of the invention, wherein the cell is preferably genetically modified and exhibits enhanced expression of the peptide siderophore in comparison to an unmodified cell.

A further aspect of the invention relates to a composition comprising a peptide siderophore and/or a bacterial cell expressing a peptide siderophore as described herein.

In a preferred embodiment, the composition comprises the peptide siderophore as described herein in its corresponding iron-complex.

A further aspect of the invention relates to a method for promoting plant growth comprising administering a peptide siderophore, or a composition, or a bacterial cell expressing a peptide siderophore as described herein, in an amount effective to promote growth of said plant.

In a preferred embodiment of the invention the method is characterized in that the bacterial cell expressing a peptide siderophore comprises a nonribosomal peptide synthetase (NRPS) gene cluster (grb), comprising preferably associated genes encoding a TonB-dependent siderophore receptor and an iron-hydroxamate transporter ATP binding protein.

In a preferred embodiment of the invention the method is characterized in that the bacterial cell expressing a peptide siderophore is selected from a Burkholderia species, preferably Burkholderia graminis, Burkholderia sp. CCGE1001, Burkholderia sp. CCGE1003, Burkholderia sp. HB1, Burkholderia phenoliruptrix BR3459a or Burkholderia kururiensis M130.

A further aspect of the invention relates to a method for promoting root growth or development, improving stress tolerance and/or for increasing crop yields of a plant, comprising administering a peptide siderophore or a composition as described herein, or a bacterial cell expressing a peptide siderophore as described herein, in an amount effective to promote root growth, stress tolerance and/or for increasing crop yields of said plant.

A further aspect of the invention relates to a method for delivering nitric oxide (NO) to a plant, comprising administering a peptide siderophore or a composition as described herein, or a bacterial cell expressing a peptide siderophore as described herein, in an amount effective to deliver NO to said plant.

A further aspect of the invention relates to a method for enhancing chlorophyll production in a plant, comprising administering a peptide siderophore or a composition as described herein, or a bacterial cell expressing a peptide siderophore as described herein, in an amount effective to enhance chlorophyll production in said plant.

In one embodiment a method is provided, wherein the plant is rice, maize or wheat.

Further embodiments of the invention are disclosed in the detailed description below.

### DETAILED DESCRIPTION OF THE INVENTION

Iron is essential for almost all life for processes such as respiration and DNA synthesis and plays a significant role in plant growth. Despite being one of the most abundant elements in the Earth's crust, the bioavailability of iron in many environments, such as the soil, is limited by the low solubility of the Fe3+ ion. This is the predominant state of iron in aqueous, non-acidic, oxygenated environments.

Siderophores are iron-chelating compounds, preferably of high-affinity, typically secreted by microorganisms such as bacteria and fungi. Siderophores may also be chemically synthesized according to established techniques. In preferred embodiments, siderophores form a stable, hexadentate, octahedral complex preferentially with Fe3+. Preferably the siderophores comprise three bidentate ligands per molecule, forming a hexadentate complex and causing a smaller entropic change than that caused by chelating a single ferric ion with separate ligands.

Ligands are considered ions or molecules that bind to a central metal atom to form a coordination complex. The bonding with the metal generally involves donation of one or more of the ligand's electron pairs. The nature of metal-ligand bonding can range from covalent to ionic. Preferred ligands of the present invention are those that bind iron, for example (without limitation) amino acid side chains with one or more hydroxy carboxylic acid, hydroxamate, catecholate and/or salicylate ligands, preferably a hydroxy carboxylic acid group.

The siderophore of the present invention may in some embodiments be produced via culturing of a bacteria that expresses and secretes the siderophore of the invention and subsequent isolation. Suitable techniques for assessing expression, detection and isolation of the siderophore are presented in the examples below and are known to a skilled person. The siderophore of the present invention may in some embodiments be produced via synthetic chemical techniques. Appropriate techniques for producing peptide siderophores via chemical synthesis are known to a skilled person (refer Peptide siderophores, Drechsel and Jung, J. Peptide Sci., Volume 4, Issue 3, May 1998, Pages 147-181).

A “peptide siderophore” refers to a molecule with siderophore function (iron-chelating function) and comprising one or more amino acid or peptide structures, or structures similar to an amino acid or peptide, for example amino acid or peptide mimetics, amino acid or peptide analogues, or other structures, either natural or synthetic, that resemble the structure and/or function of an amino acid and/or peptide. References to amino acids or peptides therefore encompass amino acids or peptide analogues or mimetics, natural or synthetic, with a similar or analogous function.

### Sequences of the Invention:

### X1-X2-X3-Gra1-X4-Gra2 (SEQ ID NO: 1)

otherwise represented as XXXZXZ (SEQ ID NO: 1), wherein X is the equivalent of any unknown, potentially variable or modified amino acid, and Z is a modified amino acid, in this case preferably Gra1 or Gra2, which may be the same or different and are amino acids comprising a side chain with an N-nitroso-hydroxylamine ligand,
wherein preferably:
a. X1 to X4 are amino acids and wherein at least one of X1 to X4 comprises a side chain with at least two atoms capable of forming one or more Fe-chelating ligands, preferably a side chain with N-nitroso-hydroxylamine, hydroxy carboxylic acid, hydroxamate, catecholate, or salicylate ligands; and
b. Gra1 and Gra2 may be the same or different and are amino acids comprising a side chain with an N-nitroso-hydroxylamine ligand.

### Asp-Thr1-Thr2-Gra1-Gly-Gra2 (SEQ ID NO: 2)

otherwise represented as DTTZGZ (SEQ ID NO: 2), Z is a modified amino acid, in this case Gra1 or Gra2, which may be the same or different and are amino acids comprising a side chain with an N-nitroso-hydroxylamine ligand,
wherein preferably Asp comprises a side chain with an additional hydroxy group adjacent to the carboxylic acid group.

### X2-X3-Gra1-X4-Gra2 (SEQ ID NO: 3)

otherwise represented as XXZXZ (SEQ ID NO: 3), wherein X is the equivalent of any unknown, potentially variable or modified amino acid, and Z is a modified amino acid, in this case preferably Gra1 or Gra2, which may be the same or different and are amino acids comprising a side chain with an N-nitroso-hydroxylamine ligand,
wherein preferably:
c. X2 to X4 are amino acids and wherein at least one of X2 and/or X3 comprises a side chain with at least two atoms capable of forming one or more Fe-chelating ligands, preferably a side chain with N-nitroso-hydroxylamine, hydroxy carboxylic acid, hydroxamate, catecholate, or salicylate ligands; and
d. the side chain of X2 and the C-terminus of Gra2, or the N-terminus of X2 and the C-terminus of Gra2, are linked to form a cyclic peptide.

A nitroso group corresponds to R1R2-N═O, wherein R1R2 are any given organic moiety, eg H, amine or alkyl. N-Nitroso groups correspond to R1R2N—N═O. A hydroxylamine group corresponds to R1R2N—OH. N-nitroso-hydroxylamine is represented by the following formula:

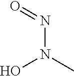

A hydroxy carboxylic acid relates to any carboxylic acid with at least one hydroxy group. Preferred ligands comprising a hydroxy carboxylic acid exhibit a hydroxy group in the alpha or beta position relative to the carboxylic acid, ie. with 1 or 2 C atoms separating the OH and COOH.

A hydroxamate is a hydroxylamine compound containing a CONOH group, well known in the field to serve as chelating agents. Derivatives comprising additional organic moieties may also be encompassed.

Catecholate is typically represented by the structure C6H4O2, well known in the field to serve as chelating agents. Derivatives comprising additional organic moieties may also be encompassed.

A salicylate is a salt or ester of salicylic acid, well known in the field to serve as chelating agents. Derivatives comprising additional organic moieties may also be encompassed.

Modifications to the peptide siderophores of the present invention, which may occur through substitutions in amino acids or their side chains, are also included within the scope of the invention. Substitutions as defined herein are modifications made to the amino acids of the peptide, whereby one or more amino acids are replaced with the same number of (different) amino acids, producing a peptide which contains a different amino acid sequence than the primary peptide. The invention also encompassed insertions or deletions of amino acids, producing a peptide which contains a different number of amino acids than the primary peptide.

In some embodiments modifications to the peptides disclosed herein will not significantly alter the function of the peptide siderophore. Like additions, substitutions may be natural or artificial. It is well known in the art that amino acid substitutions may be made without significantly altering the peptide's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the peptide. Frequently, many amino acids may be substituted by conservative amino acids without deleteriously affecting the protein's function.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

With respect to the chemical compounds described herein, the term “alkyl” refers to a branched or unbranched saturated hydrocarbon group of 1 to 12 carbon atoms, such as methyl, ethyl, n-propyl, isopropyl, w-butyl, isobutyl, f-butyl, pentyl, hexyl, heptyl, and the like. Preferred alkyl groups have 1 to 12 carbon atoms, more preferably 1 to 7, or 1 to 4 carbon atoms.

The term “alkoxy” refers to a straight, branched or cyclic hydrocarbon configuration and combinations thereof, including preferably 1 to 7 carbon atoms, more preferably 1 to 4 carbon atoms, that include an oxygen atom at the point of attachment (such as O-alkyl). An example of an “alkoxy group” is represented by the formula —OR, where R can be an alkyl group, optionally substituted with an alkenyl, alkynyl, aryl, aralkyl, cycloalkyl, halogenated alkyl, or heterocycloalkyl group. Suitable alkoxy groups include methoxy, ethoxy, n-propoxy, i-propoxy, n-butoxy, i-butoxy, sec-butoxy, cyclohexyloxy, and the like.

“Carboxyl” refers to a —COOH group. Substituted carboxyl refers to —COOR where R is aliphatic, heteroaliphatic, alkyl, heteroalkyl, or a carboxylic acid or ester.

The term “hydroxyl” is represented by the formula —OH.

The siderophores and methods of the present invention are intended to promote plant growth, or for promoting root growth or development, improving stress tolerance and/or for increasing crop yields of a plant, (e.g. crops such as fruit (e.g., strawberry), vegetable (e.g., tomato, squash, pepper, eggplant), or grain crops (e.g., soy, maize, wheat, rice, corn) tree, flower, ornamental plant, shrub (e.g., cotton, rose), bulb plant (e.g, onion, garlic) or vine (e.g., grape vine) and also, in particular, promote uptake of iron from the siderophores, promote chlorophyll production and/or promote nitric oxide provision.

In a related aspect, a method is provided for promoting growth in a plant, or for promoting root growth or development, improving stress tolerance and/or for increasing crop yields of a plant, (e.g. crops such as fruit (e.g., strawberry), vegetable (e.g., tomato, squash, pepper, eggplant), or grain crops (e.g., soy, maize, wheat, rice, corn), tree, flower, ornamental plant, shrub (e.g., cotton, rose), bulb plant (e.g, onion, garlic) or vine (e.g., grape vine) with an amount of a composition containing one or more of the siderophores described herein which modulate and in particular promote growth by, for example, improving uptake of iron by the plants from the siderophores, promote chlorophyll production and/or promote nitric oxide provision.

Suitable methods for determining improved growth, root growth or development, stress tolerance and/or crop yields are known to a skilled person or described below in the examples.

Compositions of the invention comprise in some embodiments the siderophore of the present invention, or the siderophore in its corresponding iron complex, bacterial cells, bacterial cultures, whole cell broths, liquid cultures, culture supernatants, or suspensions of or derived from bacterial strains expressing the siderophore of the present invention.

Preferred bacterial strains, without limitation, relate to Burkholderia sp., such as Burkholderia graminis, Burkholderia sp. CCGE1001, Burkholderia sp. CCGE1003, Burkholderia sp. HB1, Burkholderia phenoliruptrix BR3459a or Burkholderia kururiensis M130.

The compositions can be formulated in any manner. Exemplary formulations include but are not limited to emulsifiable concentrates (EC), wettable powders (WP), soluble liquids (SL), aerosols, ultra-low volume concentrate solutions (ULV), soluble powders (SP), microencapsulates, water-dispersed granules, flowables (FL), microemulsions (ME), nano-emulsions (NE), etc. In any formulation described herein, percent of the active ingredient is within a range of 0.0001% to 99.9999%.

The active ingredient may be considered, without limitation, to be the siderophore of the present invention, or the siderophore in its corresponding iron complex, bacterial cultures, whole cell broths, liquid cultures, or suspensions of or derived from bacterial strains expressing the siderophore of the present invention.

The compositions can be in the form of a liquid, gel or solid. A solid composition can be prepared by suspending a solid carrier in a solution of active ingredient(s) and drying the suspension under mild conditions, such as evaporation at room temperature or vacuum evaporation at 65° C. or lower.

A composition can comprise gel-encapsulated active ingredient(s). Such gel-encapsulated materials can be prepared by mixing a gel-forming agent (e.g., gelatin, cellulose, or lignin) with a culture or suspension of live or inactivated bacterial cells, or with a cell-free filtrate or cell fraction, or with a spray- or freeze-dried culture, cell, or cell fraction of a relevant bacterial strain.

Mixtures of the disclosed compositions with, for example, a solid or liquid adjuvant are prepared in known manner. For example, mixtures can be prepared by homogeneously mixing and/or grinding the active ingredients with extenders such as solvents, solid carriers and, where appropriate, surface-active compounds (surfactants). The compositions can also contain additional ingredients such as stabilizers, viscosity regulators, binders, adjuvants as well as fertilizers or other active ingredients in order to obtain special effects.

The composition can additionally comprise a surfactant to be used for the purpose of emulsification, dispersion, wetting, spreading, integration, disintegration control, stabilization of active ingredients, and improvement of fluidity or rust inhibition. The choice of dispersing and emulsifying agents, such as non-ionic, anionic, amphoteric and cationic dispersing and emulsifying agents, and the amount employed, is determined by the nature of the composition and the ability of the agent to facilitate the dispersion of the compositions.

The compositions set forth above can be combined with another agent, microorganism and/or pesticide (e.g. nematicide, bactericide, fungicide, acaricide, insecticide). Additional plant growth enhancing agents may also be combined.

The compositions disclosed herein, or formulated product, can be used alone or in combination with one or more other additional components, such as growth promoting agents and/or anti-phytopathogenic agents in a tank mix or in a program (sequential application called rotation) with predetermined order and application interval during the growing season. When used in a combination with the above-mentioned products, for example at a concentration lower than recommended on the product label, the combined efficacy of the two or more products (one of which is the said composition disclosed herein) is, in certain embodiments, greater than the sum of each individual component's effect.

### Methods of Administration

The compositions can be applied using methods known in the art. Specifically, these compositions are applied to and around plants or plant parts. Plants are to be understood as meaning in the present context all plants and plant populations such as desired and undesired wild plants or crop plants (including naturally occurring crop plants).

Crop plants can be plants which can be obtained by conventional plant breeding and optimization methods or by biotechnological and genetic engineering methods or by combinations of these methods, including transgenic plants and plant cultivars protectable or not protectable by plant breeders' rights.

Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. Plant parts also include harvested material, and vegetative and generative propagation material, for example cuttings, tubers, rhizomes, offshoots and seeds.

Treatment of plants and plant parts with the compositions set forth above can be carried out directly or by allowing the compositions to act on a plant's surroundings, habitat or storage space by, for example, immersion, spraying, evaporation, fogging, scattering, painting on, or injecting.

The compositions disclosed herein can also be applied to soil using methods known in the art. These include but are not limited to (a) drip irrigation or chemigation; (b) soil incorporation; (c) soil drenching; (d) seed treatment and dressing; (e) bare root dip; or (f) spraying of plants. The composition can be applied by root dip at transplanting, specifically by treating a fruit or vegetable with the composition by dipping roots of the fruit or vegetable in a suspension of said composition prior to transplanting the fruit or vegetable into the soil. Alternatively, the composition can be applied by spray, drip or other irrigation system. In yet another embodiment, the composition can be added as an in-furrow application.

Plant-bacterial interactions in the rhizosphere are important determinants of soil fertility and plant health. Free living bacteria that are beneficial to plant growth are known as plant growth promoting rhizobacteria (PGPR). These bacteria may be administered in combination with the compositions of the present invention. Generally, plant growth promoters function in one of multiple ways: by synthesizing plant growth regulators, by facilitating the uptake of soil nutrients, such as iron, and/or by preventing plant disease. Therefore, the effects of PGPRs can be both direct and indirect. Indirect plant growth promotion can involve antagonistic effect against phytophatogens. The PGPR may also lead to enhanced production of the siderophore of the present invention.

### FIGURES

The invention is further described by the following figures. These are not intended to limit the scope of the invention, but represent preferred embodiments of aspects of the invention provided for greater illustration of the invention described herein.

FIG. 1. Biosynthetic origin and structure of gramibactin. (A), Typical ligand systems in siderophores compared to the novel N-nitroso-hydroxylamine chelator. (B), Homologous gene clusters coding for an NRPS machinery and transport-related proteins from B. graminis and other plant-associated or soil-derived Burkholderia spp. (asterisk indicates genes coding for TonB receptor proteins). The map indicates locations where the respective strains were isolated. (C), Domain architecture of the deduced NRPS with predicted substrates of adenylation domains for all homologous assembly lines. FAL, fatty acid acyl ligase; C, condensation; A, adenylation; TE, thioesterase; green, thiolation; FA, fatty acid; n. d., domain not present in respective cluster; Gra, novel amino acid named graminine. (D), Isotope patterns and characteristic mass differences (−3H+Fe) between two observed ions indicate an iron-binding compound. (E), CAS agar plate shows B. graminis wild type and Δgrbl knockout mutant deficient in siderophore production. HPLC-DAD analysis proves absence of gramibactin in the supernatant of Δgrbl mutant. GBT, gramibactin. (F), ¹⁵N HMBC couplings measured in ¹⁵N-labeled gramibactin led to the identification of N-nitroso-hydroxylamine functions (CD₃NO₂ was used as reference). (G), HRMS-MS spectra reveal multiple losses of NO from the parent ion m/z 835.3779 [M+H]⁺. (H), Structure of gramibactin (1) with N-nitroso-hydroxylamine moieties highlighted in red.

FIG. 2. Characterization of metal-gramibactin complexes. (A), Isotope patterns (measured in negative mode) of gramibactin and its gallium complex prove existence of a LC-stable metal complex; ¹H NMR spectra show complexation-induced shifts of indicated signals of protons close to the metal-binding sites. (B), Calculated 3D model of Fe-gramibactin (calculated with Perkin Elmer Chem3D Pro 13). Hydrogen atoms omitted for better visibility. Gray: carbon; blue: nitrogen; red: oxygen; green: iron. (C), Spectrophotometric titration of gramibactin (GBT) (C_{GBT}=43 μm) shows shift of absorption maxima in dependence of pH, a characteristic feature of N-nitroso-hydroxylamines. (D), Speciation diagram of gramibactin (GBT) vs pH in I=0.1 m KCl and T=25.0±0.1° C. c_{GBT}=1 mm. (E), Speciation diagram of Fe³⁺/gramibactin (GBT) system: fraction of Fe versus pH, in I=0.1 m KCl and T=25.0±0.1° C. C_{GBT}=C_{Fe}=1 mm.

FIG. 3. Function of gramibactin in planta. (A), Photometrically determined chlorophyll contents per mg fresh weight of the 3^{rd} leaf of 21 d old corn plants grown hydroponically with solvent (control, n=6) or 50 μm EDTA (n=4), Fe-EDTA (n=6), gramibactin (n=6) or Fe-gramibactin (n=6). Bar fillings show the observed phenotype of the measured leaves. Squares represent individual data points; means are plotted together with standard deviation. ns, not significant; **P<0.01; ***P<0.001. (B), PET-CT image of 21 d old corn plants using ⁶⁸Ga³⁺ as tracer. Plants were measured for 1 h after 1 h incubation with ⁶⁸Ga-gramibactin or ⁶⁸Ga³⁺. (C), Fluorescence signals of naphtotriazole formed by reaction of released nitric oxide with 2,3-diaminonaphthalene. Squares indicate individual data points; root prot., extracted proteins from corn roots; ***P<0.001. (D), Fluorescence microscope images of cross sections from corn roots treated with 4,5-diaminofluoresceine diacetate (DAF-2DA) as probe for NO release. Fluorescence indicates presence of 4,5-diaminofluoresceine triazole (DAF-2T) formed upon NO release. After incubation with DAF-2DA roots were incubated with nutrient solution (control), 100 μm Fe-gramibactin, or 100 μm gramibactin before they were sliced. (E), Model of nitric oxide release from N-nitroso-hydroxylamines in the presence of peroxidase and H₂O₂. (12)

FIG. 4. Structures of known natural products bearing N-nitroso-hydroxylamine moieties.

FIG. 5. The PCR confirmation using primer pair Bg-NRPS-fw3 and Bg-NRPS-rv3 and template DNAs of B. graminis C4D1M Dgrbl mutant (1), wild type (2), and pGEM-Dgrbl (3). The estimated size of amplicons; lanes 1 and 3; 1,372 bp, 2; 274 bp. M; marker.

FIG. 6. Schematic analysis of adenylation domains for (A), the predicted amino acid (p-hydroxyphenylglycine, Table 2), (B), graminine, (C), NhOrn and (D-F), other Orn derivatives using the simplified model by Stachelhaus et al. (16) Acidic (light blue), basic (red), non-polar aliphatic (orange), polar and uncharged (light green), aromatic (purple) amino acids.

FIG. 7. Key COSY and HMBC correlations of gramibactin in DMSO-d₆.

FIG. 8. Isotope patterns of (A), gramibactin and (B), ¹⁵N-labeled gramibactin. (C), Analysis of isotope pattern regarding number of incorporated ¹⁵N. Colors indicate the number of ¹⁵N within the molecule and their contribution to the isotope pattern.

FIG. 9. Isotope patterns of (A), gramibactin and (B), partially deuterated gramibactin. (C), Analysis of isotope pattern with regard to the number of incorporated ²H. Colors indicate the number of ²H within the molecule and their contribution to the isotope pattern.

FIG. 10. Key experiments to elucidate the stereochemistry of threonines and graminines. (A), LCMS profiles (TIC in negative mode) of Marfeys derivatives of hydrolysed graminines obtained after hydrolysis of gramibactin in HCl/H₂O and DCl/D₂O together with isotope patterns of highlighted peaks. 1 corresponds to the derivative of hydrolysed 1-graminine and 2 to the one of d-graminine. (B), ¹H NMR signals of selected amide and methyl protons in the native gramibactin and (C), in gramibactin with incorporated l-threonine-2,3-d₂.

FIG. 11. Representative potentiometric titration curves of gramibactin (solid line, c_{gramibactin}=1.26 mm) and gramibactin/Fe³⁺ system at 1:1 ratio (dashed line, c_{gramibactin}=c_{Fe}=0.87 mm) in I=0.1 m KCl and T=25.0±0.1° C.

FIG. 12. Experimental absorption spectra of (A), gramibactin (GBT) and (B), Fe³⁺/gramibactin (GBT) system at 1:2 ratio, measured at different pH values, C_{GBT}=0.3 mmol dm⁻³, in l=0.1 m KCl and T=25.0±0.1° C. Calculated molar absorbance spectra for the individual species (C), gramibactin and (D), Fe³⁺/gramibactin system.

FIG. 13. Obtained fluorescence signals of naphtotriazole formed by reaction of released nitric oxide with 2,3-diaminonaphthalene. The probe was incubated with the respective components and fluorescence was measured using a microplate reader. root prot., extracted proteins from corn roots. (A-D) represent each one individual experiment using root protein extracts from a different corn plant. Assays were performed in triplicates and every replicate was measured three times. All values were normalized (highest mean set to 100) and means were plotted together with standard deviation.

FIG. 14. IR spectrum of gramibactin.

FIG. 15. ¹H spectrum of gramibactin (DMSO-d₆, 500 MHz, 298 K).

FIG. 16. ¹³C spectrum of gramibactin (DMSO-d₆, 125 MHz, 298 K).

FIG. 17. DEPT spectrum of gramibactin (DMSO-d₆, 125 MHz, 298 K).

FIG. 18. COSY spectrum of gramibactin (DMSO-d₆, 500 MHz, 298 K).

FIG. 19. HSQC spectrum of gramibactin (DMSO-d₆, 500 MHz, 298 K).

FIG. 20. HMBC spectrum of gramibactin (DMSO-d₆, 500 MHz, 298 K).

FIG. 21. ¹⁵N-HMBC spectrum of ¹⁵N-labeled gramibactin (DMSO-d₆/CD₃NO₂ 3:1, 500 MHz, 298 K) (spectrum is calibrated on CD₃NO₂ as an internal reference).

FIG. 22. ¹H spectrum of Cbz-l-graminine (4) (DMSO-d₆, 500 MHz, 298 K).

FIG. 23. ¹³C spectrum of Cbz-l-graminine (4) (DMSO-d₆, 125 MHz, 298 K).

FIG. 24. DEPT spectrum of Cbz-l-graminine (4) (DMSO-d₆, 125 MHz, 298 K).

FIG. 25. COSY spectrum of Cbz-L-graminine (4) (DMSO-d₆, 500 MHz, 298 K).

FIG. 26. HSQC spectrum of Cbz-L-graminine (4) (DMSO-d₆, 500 MHz, 298 K).

FIG. 27. HMBC spectrum of Cbz-L-graminine (4) (DMSO-d₆, 500 MHz, 298 K).

FIG. 28. ¹⁵N-HMBC spectrum of Cbz-L-graminine (4) (DMSO-d₆/CD₃NO₂ 3:1, 500 MHz, 298 K) (spectrum is calibrated on CD₃NO₂ as an internal reference).

FIG. 29. ¹H spectrum of gramibactin with incorporated l-threonine-2,3-d₂ (DMSO-d₆, 500 MHz, 298 K).

FIG. 30. ¹H spectrum of Ga-gramibactin (DMSO-d₆, 600 MHz, 298 K).

FIG. 31. ¹³C spectrum of Ga-gramibactin (DMSO-d₆, 150 MHz, 298 K).

FIG. 32. DEPT spectrum of Ga-gramibactin (DMSO-d₆, 150 MHz, 298 K).

FIG. 33. COSY spectrum of Ga-gramibactin (DMSO-d₆, 600 MHz, 298 K).

FIG. 34. HSQC spectrum of Ga-gramibactin (DMSO-d₆, 600 MHz, 298 K).

FIG. 35. HMBC spectrum of Ga-gramibactin (DMSO-d₆, 600 MHz, 298 K).

FIG. 36. Synthesis of Cbz-l-graminine (4). For experimental details see Methods section.

### EXAMPLES

The invention is further described by the following examples. These are not intended to limit the scope of the invention, but represent preferred embodiments of aspects of the invention provided for greater illustration of the invention described herein.

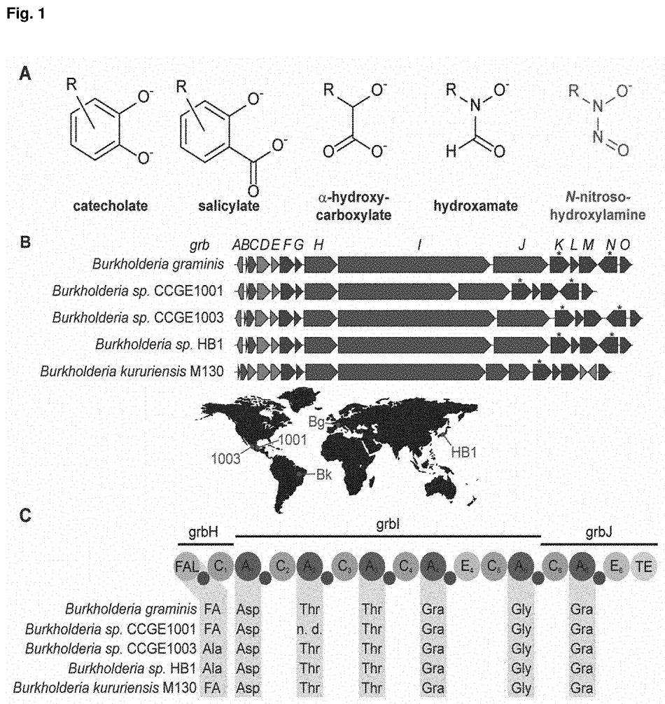

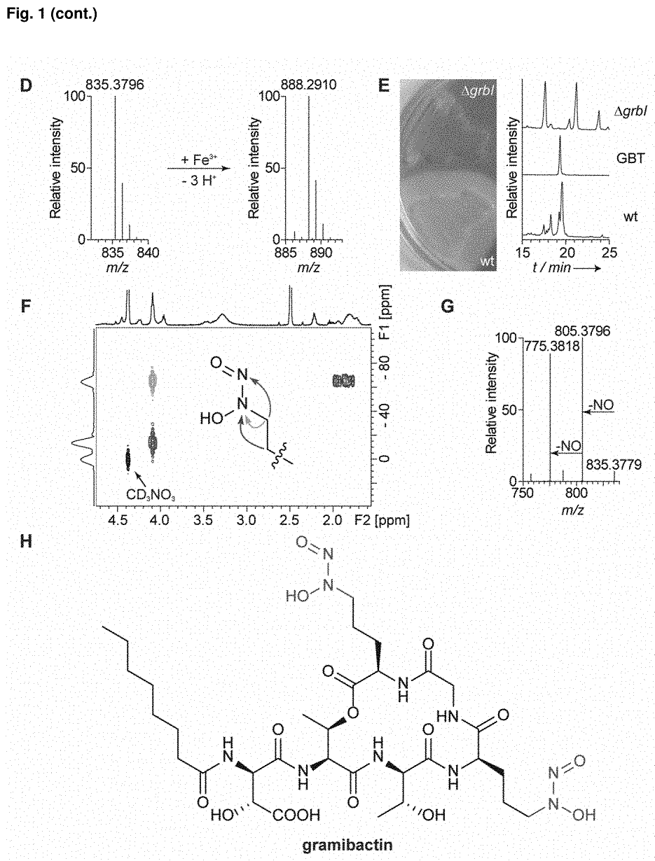

To discover structurally novel siderophores produced by rhizosphere bacteria, we initially investigated Burkholderia graminis, a species isolated from the rhizospheres of maize and wheat, two of the top three cereal crop plants by tonnage worldwide (4). We mined the genome sequence of the maize isolate (strain C4D1M) for signature genes for siderophore biosynthesis, transporters and receptors. Our bioinformatic analysis revealed the presence of a nonribosomal peptide synthetase (NRPS) gene cluster (grb) comprising associated genes coding for a TonB-dependent siderophore receptor (5) and an iron-hydroxamate transporter ATP binding protein (Table 1). Surprisingly, we discovered highly homologous, yet cryptic, gene clusters in the genomes of various related soil-borne or rhizosphere colonizing bacteria, such as Burkholderia sp. CCGE1001 (6) and CCGE1003 (6), Burkholderia sp. HB-1 (7), and Burkholderia kururiensis M130 (8), isolated from distinct locations all over the world (FIG. 1B).

A more detailed analysis of the NRPS including the substrate prediction of the adenylation domains suggested that the encoded metabolite is an N-acylated polypeptide (FIG. 1C) composed of aspartate, threonine and glycine. Intriguingly, two non-canonical amino acid moieties remained unpredictable using the commonly used algorithm. The same amino acid signature was deduced from the orphan NRPS genes of the related rhizosphere bacteria.

To detect the putative siderophore, B. graminis was cultured in a variety of media and tested for iron-chelating potency using the CAS assay. By LC-HRESIMS analysis of the culture broth extract from liquid MM9 medium, we detected two species with m/z 888.2910 [M+H]⁺ and m/z 835.3793 [M+H]⁺, for which the elemental compositions of C₃₂H₅₅O₁₆N₁₀ (calc. m/z 835.3792) and C₃₂H₅₂O₁₆N₁₀Fe (calc. m/z 888.2907) were deduced (FIG. 1D). Since the masses differed only in the mass of one equivalent of iron and three protons liberated by complexation, we concluded that the two species represented an iron-chelating compound, named gramibactin (1), and its corresponding iron complex.

To confirm that the identified gene cluster is responsible for gramibactin production, we generated an NRPS-deficient mutant by disruption of grbl by inserting a chloramphenicol resistance cassette. Cultures of B. graminis Δgrbl showed no color change when grown on CAS agar plates, and no gramibactin could be detected in liquid culture supernatants (FIG. 1E). Thus, the grb gene locus was unequivocally linked to the production of a siderophore.

Sufficient amounts for a full structure elucidation of gramibactin were obtained from a scaled-up fermentation using an adsorber resin. Purification was achieved by repeated preparative RP-HPLC. MS fragmentation and 1 D-/2D-NMR experiments in combination with chemical degradation, derivatisation, and isotope labeling experiments revealed the cyclic lipodepsipeptide architecture of gramibactin composed of octanoic acid, glycine, D-threo-beta-hydroxyaspartate and two threonine moieties (L-threo and D-allo) (see Methods). The carbon backbone of the remaining two amino acids was elucidated based on their spin systems in combination with HMBC couplings to be the same as in ornithine (Orn). However, the downfield-shifted ¹H and ¹³C signals of the δ-methylene units (δ_{c} 60.8 & 61.3 ppm; δ_{H} 4.07 & 4.09 ppm) indicated the presence of a strongly electron-withdrawing group in lieu of the ω-amino moiety of Orn. According to exact mass analyses the amino acid residues are rich in nitrogen since only six out of ten nitrogen atoms are located in the peptide backbone.

To elucidate the structure of the nitrogen-rich side chain, (¹⁵NH₄)₂SO₄ was added to a B. graminis culture, and the ¹⁵N-enriched gramibactin was isolated. ¹⁵N-HMBC experiments showed two chemically different nitrogen species in each of the Orn-like side chains, with ¹⁵N-¹H couplings to H_{γ} and Hδ, and Hδ, respectively (FIG. 1F). Characteristic losses of masses corresponding to NO in HR-MS² fragmentations (FIG. 1G) and a positive Griess test (9) suggested that gramibactin harbors two N-nitroso moieties. The identity of the nitrosylated hydroxylamine was corroborated by typical pH-dependent shifts in the UV spectra (λmax=226 at pH 2 to λmax=248 at pH 10) and diagnostic IR bands at 1,457 cm⁻¹, 1,055 cm⁻¹ and 960 cm⁻¹. Thus, gramibactin features an as to yet unknown amino acid, which was named graminine (Gra).

Finally, the structure of graminine was proven by total synthesis of the protected amino acid (see Methods) and comparison of ¹⁵N chemical shifts in ¹⁵N-HMBC experiments. In accordance with the in silico analysis of the NRPS domains, the absolute configuration of the graminine building blocks was determined to be D by a modified Marfey approach involving the MS-based detection of deuterium exchange in the event of epimerization during peptide hydrolysis using synthesized Cbz-L-graminine (4) as a reference (see Methods). Thus, the structure of gramibactin was fully elucidated (FIG. 1H).

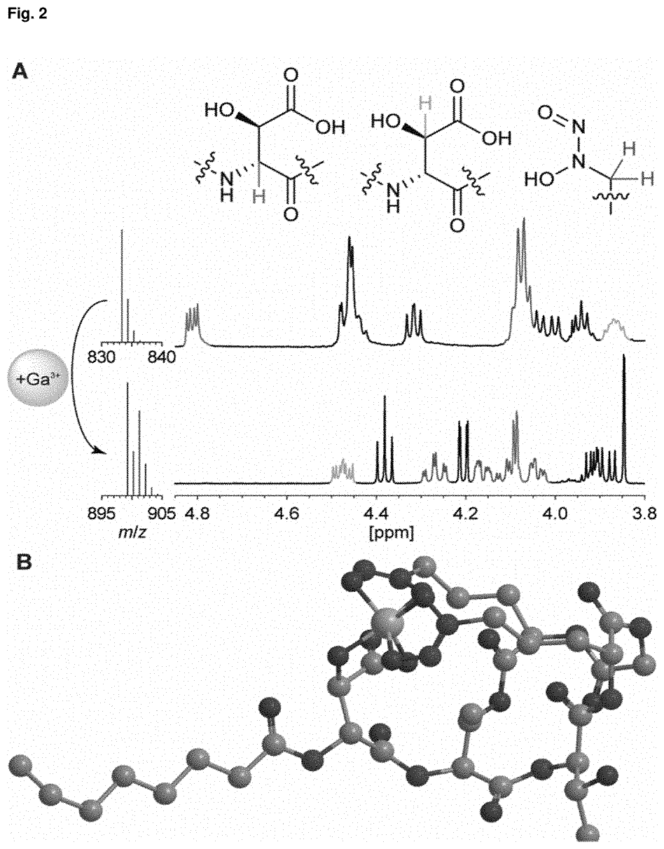

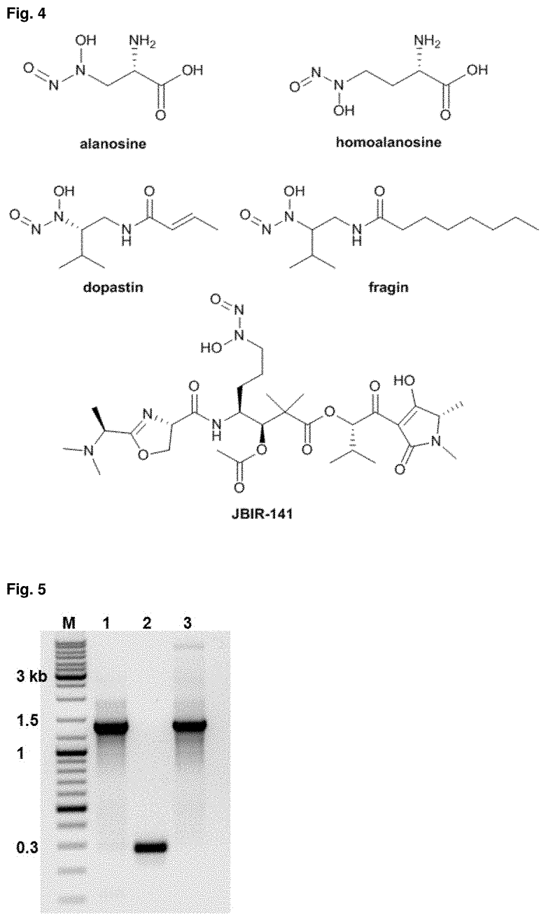

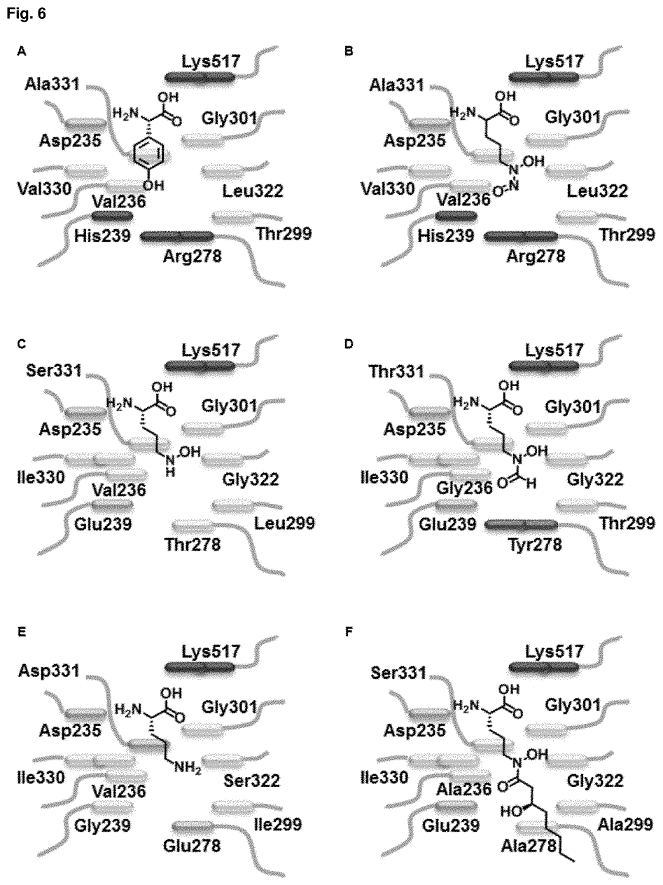

N-Nitroso-hydroxylamines (or diazeniumdiolate groups) are extremely scarce in nature, with only a handful of examples out of 300,000 structures that have been reported in the literature (FIG. 4). It should be highlighted that chelating properties of N-nitroso-hydroxylamines, which are isosteric to hydroxamates, have only been reported for synthetic compounds, such as the copper-complexing reagent cupferron (10). In natural siderophores, this functional group has been fully unprecedented. To test whether the nitroso residues are directly involved in complex formation, we prepared the NMR-compatible, iron-mimicking gallium(III) complex of gramibactin. Based on complexation-induced changes of chemical shifts in the proton spectra, the beta-hydroxy carboxylic acid and both N-nitroso-hydroxylamine moieties were assigned as the ligand system providing six coordination sites. The loss of chemical equivalence of the methylene protons in the graminine side chains (FIG. 2A) can be explained by the cage-like structure of the gallium complex, which leads to a loss of flexibility in the aliphatic amino acid residue (FIG. 2B).

To evaluate the actual strength of the siderophore, we conducted potentiometric and spectrophotometric titrations to evaluate the acid/base behavior of gramibactin and to determine the complex formation constant of the iron complex (FIG. 2C). We found that gramibactin releases four protons upon metal complexation, which supports the binding model. Using the obtained pKₐ values it was possible to construct a speciation diagram of the stepwise deprotonated gramibactin species, revealing that both graminine residues are deprotonated at physiological pH (FIG. 2D). By a series of titrations using disodium EDTA as a competitive chelating agent we determined the log □ to be 27.6±0.1, which is comparable to the power of bacterial hydroxamate siderophores (1) forming highly stable Fe^{III} complexes over a wide range of pH (FIG. 2E). Nonetheless, gramibactin does not appear to be a typical siderophore, which is only formed under iron-limitation, as surprisingly high titers were produced independent of Fe^{III} concentration. It thus appeared conceivable that the rhizosphere bacteria excrete the metal chelator to provide the host plant with iron.

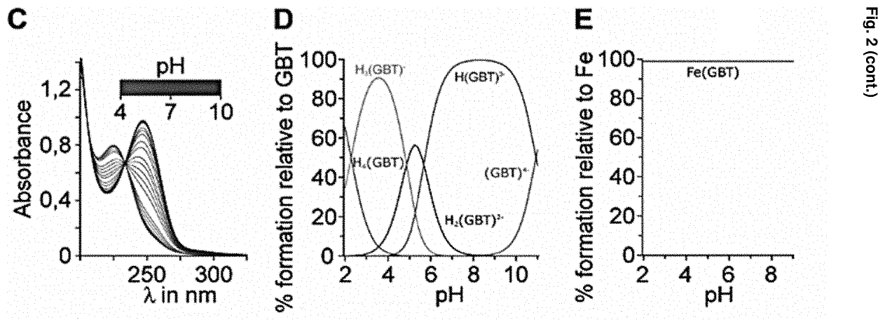

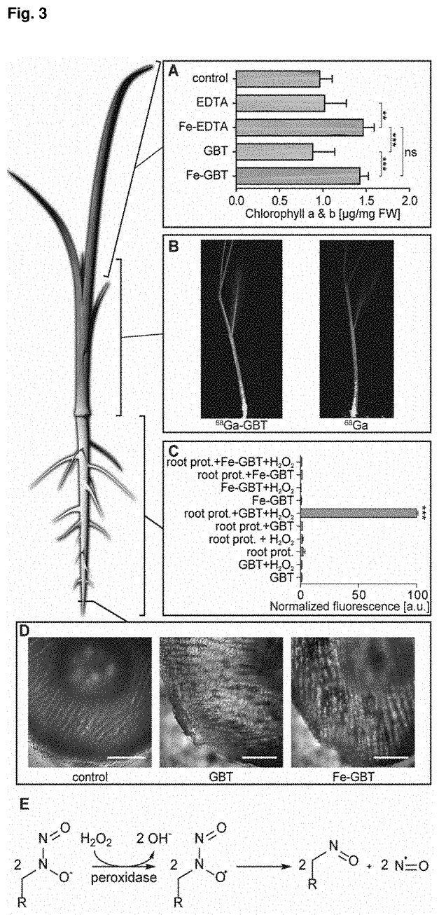

To test whether the host plant is capable of taking up metals bound to gramibactin we performed PET/CT experiments using ⁶⁸Ga as tracer nuclide, which decays into ⁶⁸Zn with positron emission. As in the NMR complexation experiments, Ga^{III} was used as a surrogate since it has comparable properties to iron in terms of complexation, yet ⁶⁸Ga is easier to generate and handle than ⁵²Fe, the only feasible positron-emitting iron isotope. This isotope would have a suitable half-life (˜8h), but decays into ⁵²Mn, which is another positron emitter that interferes with the measurement. The radiolabel was administered to hydroponically grown corn plants after replacing the nutrient solution and thoroughly washing the roots to remove phytosiderophores. After incubation with the radioactive-labeled nutrient solution the plant was subjected to PET/CT to monitor and compare uptake and spatial distribution of gallium bound to gramibactin with the uptake of unbound gallium. By LC coupled to a radio detector we evaluated the stability of the Ga-gramibactin complex at different pH values and in the nutrient solution used for administering the radioactive label to the plant. The activity was measured in the shoot following both treatments (FIG. 3B). In addition, we determined and compared standard uptake values. As these values do not differ for unbound and chelated metal, the presence of gramibactin does not impair the metal uptake of the plant.

To examine whether the plant can actually utilize Fe bound to gramibactin we performed in vivo experiments. Corn plants were grown in nutrient solutions deficient in iron, or supplementing either Fe-EDTA (positive control) or Fe-gramibactin. Solvent and metal-free chelators were used as negative controls. As readout for iron uptake we measured the chlorophyll content of youngest leaves, since chlorophyll biosynthesis is strongly dependent on iron availability (11). Leaves of plants grown only in the presence of the solvent or the metal-free ligands showed a much paler color compared to plants treated with iron-containing chelates (FIG. 3A). Quantification of extracted chlorophyll confirmed the phenotypic difference; while iron-free cultured plants contained 0.9-1.0 μg total chlorophyll per mg fresh weight of the leaf, plants supplied with the Fe chelates showed 50% higher total chlorophyll contents (1.4-1.5 μg per mg fresh weight). This dramatic difference proved that iron is effectively sequestered from gramibactin and utilized by corn plants.

Beyond Fe supply, the regulation of downstream responses to iron deficiency and overload is critical for plants. It is well known that nitric oxide is a plant hormone that mediates iron homeostasis. The structure of gramibactin strongly suggests that it could serve as an NO donor (10), as N-nitroso-hydroxylamine ligands could be capable of releasing nitric oxide upon contact with peroxidases and hydrogen peroxide (FIG. 3E) (12). Notably, these enzymes as well as reactive oxygen species are present in root tissue as they are necessary for root growth (13).

To probe the proposed enzymatic NO release in vitro, we extracted soluble proteins from ground root tissue of hydroponically grown plants into phosphate buffer. To monitor the liberation of NO in the in vitro enzyme assay, we used 2,3-diaminonaphthalene (DAN) as a reporter. Nitric oxide reacts with this chemical probe to form the 2,3-napthotriazole, and its strong fluorescence can be readily measured using a microplate reader. Extracted root proteins were incubated with the probe, hydrogen peroxide and either Fe-gramibactin or the iron-free ligand, using heat-inactivated proteins as negative control. A drastically increased fluorescence was only observed in the sample supplemented with gramibactin (FIG. 3C). Thus, gramibactin represents a novel NO donor. Interestingly, it appears that iron coordination stabilizes the N-nitroso-hydroxylamine by impairing the abstraction of an electron from the oxygen, which would be necessary for the oxidation and subsequent decay under nitric oxide release. As our in vivo experiments unequivocally showed the Fe-gramibactin complex is dissociated in the plant, which would provide the free ligands for NO liberation.

To verify the postulated NO release in planta we treated fresh corn seedlings with diaminofluoresceine-2-diacetate (DAF-2DA), a cell-permeable probe that captures NO with formation of a highly fluorescent triazole. After loading maize roots with the chemical probe they were incubated with negative controls, gramibactin, or Fe-gramibactin, then cut and inspected by fluorescence microscopy. Whereas no fluorescence could be detected in the control sample treated with the probe only, a strong signal was observed in the root treated with gramibactin (FIG. 3D). Thus, NO is in fact liberated from gramibactin in the plant tissue. A weak signal was also detectable in the root treated with the Fe-gramibactin complex. This finding is important as it confirms that iron is removed from gramibactin in vivo, thus providing free ligands that can subsequently release nitric oxide. Finally, fluorescence microscopy also allowed localizing NO, which is apparently released into the intracellular space. This observation is plausible as peroxidases, which are required for NO release, are typically located in the cell membrane.

Plants have been known to acquire iron via two main avenues, reductases and phytosiderophores (11, 14). However, recent work suggested that bacteria could play a key role in the rhizosphere as their siderophores may solubilize iron and make it accessible to the plant (3). It has been shown that the plant Fe status may affect the composition of siderophore-secreting microorganisms in the rhizosphere (15) and that uptake and translocation of iron in plants can be modulated by interacting microorganisms (3, 16). Although bacterial siderophores have been reported to extract iron from plant-derived organic matter (17), experiments with hydroponic cultures demonstrated that beyond Fe-EDTA, several bacterial and fungal Fe-siderophore complexes may represent viable sources of Fe for a variety of plants (18-21). Whereas these previous studies were limited to non-natural host-siderophore combinations, this study indicates that a siderophore of rhizosphere bacteria has a beneficial effect on the receiving crop plant. Surprisingly, this interdomain-acting molecule represents a novel type of siderophore featuring novel N-nitroso ligands for the complexation of iron. The rare N-nitroso-hydroxylamine moiety is thus an important addition to the known Fe-binding motifs in naturally occurring iron chelators and may inspire the design of related functional molecules.

The most unexpected finding is, however, that the iron-binding ligands have a second function as NO donor. The release of nitric oxide by a metabolite from rhizosphere bacteria is significant since NO represents an important plant hormone regulating many different functions in the plant, including growth, defense mechanisms and formation of symbioses (22-24). In the context of siderophores it is particularly noteworthy that NO also plays a key role in iron homeostasis and improves internal iron availability, likely by crosstalk with ferritin and frataxin, and formation of iron-nitrosyl complexes (25-28).

Our results also indicate that similar NO-donating siderophores are widespread in the plant environment, since orthologous gene clusters are shared among rhizosphere bacteria that are associated with graminaceous crop plants (4, 8) as well as with nodule-forming legumes (6). In this context it is noteworthy that exogenously supplied, synthetic NO-donors (29) were found to increase plant fitness, root growth and tolerance towards stress (30, 31). Administering siderophores on plants, on the other hand, may be an approach to control pathogens (32, 33) and to mobilize and supply iron to the crop plants (34). Thus, the discovery of a novel bifunctional siderophore-NO-donor is not only intriguing from a chemical perspective but also points to an important ecological role. Thus, gramibactin or producer strains may find application in agriculture (35).

### REFERENCES FOR THE ABOVE EXAMPLES

1. R. C. Hider, X. Kong, Nat. Prod. Rep. 27, 637 (2010).
2. R. Zhang, J. M. Vivanco, Q. Shen, Curr. Opin. Microbiol. 37, 8 (2017).
3. C. W. Jin, Y. Q. Ye, S. J. Zheng, Ann. Bot. 113, 7 (2014).
4. V. Viallard et al., Int. J. Syst. Evol. Microbiol. 48, 549 (1998).
5. N. Noinaj, M. Guillier, T. J. Barnard, S. K. Buchanan, Annu. Rev. Microbiol. 64, 43 (2010).
6. L. F. G. Zuleta et al., BMC Genomics 15, 535 (2014).
7. Y. Ohtsubo et al., Genome Announc 3, (2015).
8. B. G. Coutinho, D. Passos da Silva, J. O. Previato, L. Mendonca-Previato, V. Venturi, Genome Announc 1, e0022512 (2013).
9. F. Feigl, C. C. Neto, Anal. Chem. 28, 1311 (1956).
10. J. A. Hrabie, L. K. Keefer, Chem. Rev. 102, 1135 (2002).
11. T. K. Kobayashi, N. K. Nishizawa, Annu. Rev. Plant Biol. 63, 131 (2012).
12. T. A. Alston, D. J. Porter, H. J. Bright, J. Biol. Chem. 260, 4069 (1985).
13. A. Liszkay, E. van der Zalm, P. Schopfer, Plant Physiol. 136, 3114 (2004).
14. D. Staiger, Angew. Chem. Int. Ed. 41, 2259 (2002).
15. C. W. Jin, G. X. Li, X. H. Yu, S. J. Zheng, Ann. Bot. 105, 835 (2010).
16. G. Carrillo-Castaneda, J. J. Munoz, J. R. Peralta-Videa, E. Gomez, J. L. Gardea-Torresdey, J. Plant Nutr. 28, 1853 (2005).
17. A. M. Kraepiel, J. P. Ballenger, T. Wichard, F. M. Morel, Biometals 22, 573 (2009).
18. L. M. Chen, W. A. Dick, J. G. Streeter, J. Plant Nutr. 23, 2047 (2000).
19. G. Vansuyt, A. Robin, J. F. Briat, C. Curie, P. Lemanceau, Mol. Plant-Microbe Interact. 20, 441 (2007).
20. Z. Yehuda, M. Shenker, Y. Hadar, Y. N. Chen, J. Plant Nutr. 23, 1991 (2000).
21. W. Radzki et al., Antonie van Leeuwenhoek 104, 321 (2013).
22. S. Trevisan, A. Manoli, S. Quaggiotti, Plant Signal. Behav. 9, e28290 (2014).
23. I. Hichri et al., J. Exp. Bot. 66, 2877 (2015).
24. Y. Wang, E. G. Ruby, Cell Microbiol. 13, 518 (2011).
25. M. Graziano, L. Lamattina, Trends Plant Sci. 10, 4 (2005).
26. A. Buet, M. Simontacchi, Ann. N.Y. Acad. Sci. 1340, 39 (2015).
27. L. Ramirez, E. J. Zabaleta, L. Lamattina, Ann. Bot. 105, 801 (2010).
28. L. Ramirez, M. Simontacchi, I. Murgia, E. Zabaleta, L. Lamattina, Plant Sci. 181, 582 (2011).
29. P. G. Wang et al., Chem. Rev. 102, 1091 (2002).
30. C. M. C. P. Gouvêa, J. F. Souza, C. A. N. Magalhaês, I. S. Martins, J. Plant Growth Regul. 21, 183 (1997).
31. H. C. Oliveira, B. C. Gomes, M. T. Pelegrino, A. B. Seabra, Nitric Oxide 61, 10 (2016).
32. M. Delledonne, Y. Xia, R. A. Dixon, C. Lamb, Nature 394, 585 (1998).
33. I. J. Schalk, M. Hannauer, A. Braud, Environ. Microbiol. 13, 2844 (2011).
34. M. Saha et al., Environ. Sci. Pollut. Res. Int. 23, 3984 (2016).
35. F. D. Andreote, E. Pereira, M. C. Silva, Curr. Opin. Microbiol. 37, 29 (2017).

### Materials and Methods

### Bacterial Strains and General Culture Conditions

Burkholderia graminis wild-type strain C4D1M was obtained from the DSMZ GmbH (Braunschweig) as stock culture. B. graminis was cultured in LB medium or on agar at either 28° C. or 30° C. Chloramphenicol (34 μg mL⁻¹) was used as a selection marker for E. coli XL1 Blue or TOP10.

Preparation of Knockout Strains of B. graminis C4D1M

Genomic DNA was isolated from B. graminins C4D1M as described previously. (1) Two internal gene fragments of grbl were amplified by PCR with the primer pairs Bg-NRPS-fw/Bg-NRPS-PacI and Bg-NRPS-KpnI/Bg-NRPS-NheI using DeepVent polymerase (New England Biolabs). The PCR product containing the chloramphenicol resistance gene, which was amplified from pACYC184 with the primers Cm-fw-PacI and Cm-rv-KpnI using DeepVent polymerase. The amplicons were purified with the gel purification kit (Illustra GFX PCR DNA and Gel Band Purification Kit, GE Healthcare). Obtained amplicons were cloned into pGEM T-easy vector, and the resulting plasmids pGEM-Bg-NRPS-KO1, pGEM-Bg-NRPS-KO2, and pGEM-Cm were restricted with PacI/SpeI, KpnI/NheI, and PacI/KpnI, respectively (Table 3, Table 4). The restricted three-gene fragments were ligated by T4 DNA ligase followed by transformation into E. coli TOP10, generating pGEM-Dgrbl.

B. graminis C4D1M cells from an LB agar plate were cultured overnight at 30° C. Overnight 0.5 mL cultured cells were inoculated in 50 mL LB medium and cultured up to OD₆₀₀=0.5 at 30° C. The cultured broth was centrifuged at 4,000×g for 10 min, and the supernatant was removed. Precipitated cells were resuspended in 300 mm sucrose buffer and centrifuged. After repeating this washing step twice, the washed cells were resuspended in 300 mm sucrose buffer and subjected to electroporation (200 kV) with knockout plasmids (5-10 μg). Transformed cells were precultured in LB medium (1 mL) for 4 h at 30° C. with shaking and then plated on LB agar plates with chloramphenicol (34 μg mL⁻¹). Some positive colonies were observed 4 days later and confirmed by PCR (FIG. 5).

### MM9 Media Preparation(2, 3)

One liter of MM9 medium was prepared as follows. Solution A [350 g K₂HPO₄ and 100 g KH₂PO₄ dissolved in 1 L ddH₂O] and solution B [29.4 g NaCl, 50 g (NH₄)₂SO₄, 5 g MgSO₄ dissolved in 1 L ddH₂O] were prepared and autoclaved separately. 2 g of an amino acid mixture [2 g each of I-alanine, 1-arginine, 1-asparagin, 1-aspartate, 1-cysteine, 1-glutamine, 1-glutamate, glycine, 1-isoleucine, 1-proline, 1-serine, 1-threonine, 1-tyrosine, 1-valine, 1-phenylalanine] were dissolved in 900 mL ddH₂O and autoclaved. After cooling down, 20 mL of each solution A and B were added. Additionally, 16.7 mL of 1-leucine 100 mm, 5 mL of 1-histidine 60 mm, 10 mL of 1-lysine 100 mm, 10 mL of I-tryptophane 40 mm, 10 mL of 1-methionine 40 mm, and 20 mL of glucose 50% (w/v) were added. Optionally, 1 mL of a trace element solution [per liter: 40 mg ZnCl₂, 200 mg FeCl₃.6H₂O, 10 mg CuCl₂.2H₂O, 10 mg MnCl₂.4H₂O, 10 mg Na₂B₄O₇.10H₂O, 10 mg (NH₄)₆Mo₇O₂₄.4H₂O] was added.

### CAS Agar Preparation

One liter of CAS agar (4) was prepared as follows. To obtain CAS reagent solution, solution 1 [0.06 g chrome azurol S dissolved in 50 mL ddH₂O mixed with 0.0027 g FeCl₃.6H₂O dissolved in 10 mL 10 mm HCl] was slowly added to solution 2 [0.073 g HDTMA (1-hexadecyltrimethylammonium chloride) dissolved in 40 mL ddH₂O], and the dark blue/violet solution was autoclaved. After cooling down to ca. 55° C. it was slowly added to 950 mL of the above mentioned sterilized MM9 medium that did not contain trace element solution, but 8.06 g 1,4-piperazinediethanesulfonic acid (PIPES) and 15 g L⁻¹ agar. The pH was adjusted to 6.8 prior autoclaving the amino acid mixture. B. graminis wild-type and mutant strains were inoculated from LB agar plates and then cultured at 30° C. for 3 days.

### Siderophore Production in Liquid Media

B. graminis wild type was pre-cultured in LB medium with shaking overnight at 30° C. The obtained cultures were centrifuged and resuspended in the mentioned modified MM9 medium with added trace element solution and then inoculated to MM9 medium in baffled Erlenmeyer flasks with shaking at 28° C. for 24 hours. After cultivation, the culture broth was centrifuged at 4,000 rpm and 24° C. for 15 min. The obtained supernatant was stirred with pre-swollen XAD-16 for 1-2 hours. The resin was then filtered off, washed with H₂O and subsequently eluted with pure MeOH. The obtained crude extract was concentrated under reduced pressure and subjected to further analysis by analytical HPLC and LCMS.

### General Analytical Procedures

NMR spectra were measured on Bruker Avance DRX 500 MHz or 600 MHz spectrometers (600 MHz with cryo probe) in DMSO-d₆ or DMSO-d₆/CD₃NO₂. Spectra were referenced to the residual solvent peak. UV spectra were obtained on a Shimadzu UV-1800 spectrometer. A Jasco Fourier Transform Infrared Spectrometer 4100 was used to measure the infrared spectra using the ATR technique. For LC-MS measurements an Agilent 1100 coupled to a Bruker HCTultra PTM Discovery System with electrospray ion source using a Phenomenex Synergi 4u Hydro-RP 80A (250×4.6 mm, 4 μm) and an elution gradient [solvent A: H₂O+0.1% HCOOH, solvent B: MeCN, gradient: 0.5% to 99.5% in 30 min, flow rate 1 mL min⁻¹] was used. LC-HRMS measurements were carried out on a Thermo Fisher Scientific Exactive Orbitrap with an electrospray ion source using a Betasil 100-3 C₁₈ column (150×2.1 mm) and an elution gradient [solvent A: H₂O+0.1% HCOOH, solvent B: acetonitrile, gradient: 5% B for 1 min, 5% to 98% B in 15 min, 98% B for 3 min, flow rate: 0.2 mL min⁻¹, injection volume: 5 μL].

### Isolation of Gramibactin (6)

The obtained crude extract from a 2-L B. graminis culture was separated using a Sephadex LH-20 column. Collected fractions were analyzed by LC-MS. Fractions containing gramibactin were pooled and further fractionated by preparative HPLC using a Kromasil 100 C₁₈ column (250×20 mm, 5 μm) with an elution gradient [solvent A: H₂O+0.1% TFA, solvent B: 83% aqueous MeCN, gradient: 10 min 10% B, 10%-60% B in 20 min, flow rate 12 mL min⁻¹, Rₜ=28-29 min]. Final purification was achieved by preparative HPLC using a Phenomenex Synergi 10μ Hydro-RP 80 [250×21.2 mm, 10 μm] and gradient elution [solvent A: H₂O+0.1% TFA, solvent B: 83% aqueous MeCN, gradient: 10 min 10% B, 10%-60% B in 30 min, flow rate 12 mL min⁻¹, Rₜ=34.5 min]. The collected fraction was lyophylized to yield 18.0 mg of gramibactin as a white powder.

HRESIMS: [M+H]⁺=835.3799 (calculated for C₃₂H₅₅N₁₀O₁₆ 835.3792); IR (ATR): n=3,000 (w, n(OH)), 2,932 (w, n(CH)), 2,856 (w, n(CH)), 1,741 (w, n(C═O)), 1,653 (s, n(C═O)), 1,526 (s, n(N═O)), 1,056 (m, n(N—OH)), 961 (m, n(N—O)). NMR data see Table 6.

### Structure Elucidation of Gramibactin

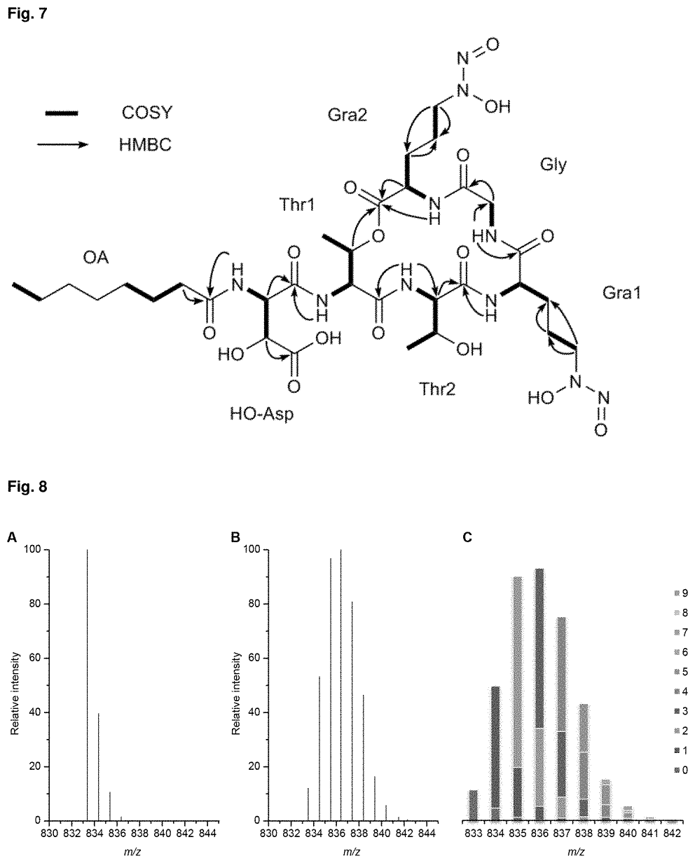

The mild hydrolysis of gramibactin in aqueous sodium hydroxide yielded a compound with m/z 851 ([M−H]⁻). Addition of the mass equivalent of water to gramibactin indicated the cleavage of a lactone ring. The presence of an ester bond explains HMBC couplings between the C-terminal carboxyl group with the proton in b-position of threonine 1 (FIG. 7) as well as the downfield shifted ¹H_{g} signal in threonine 1 compared to threonine 2 (d_{Hg} 5.21 compared to 3.94). Thus, gramibactin belongs to the group of cyclic lipodepsipeptides.

1D and 2D NMR experiments indicated the presence of six amino acids in the gramibactin backbone connected to a fatty acid (octanoic acid, OA), which is in agreement with the predicted scaffold. Based on COSY, HMBC and TOCSY spectra three amino acids were identified as threonine (twice) and glycine. From HMBC, HSQC and COSY data the carbon backbone of aspartate as a fourth amino acid was deduced. A missing proton at the b-carbon together with downfield shifted ¹³C_{b} and ¹H_{b} signals compared to aspartate (d_{C} 70.6 ppm; d_{H} 4.46 ppm) revealed that the b-position is hydroxylated.

### Griess Test (5)

For preparing Griess' reagent, 5 mL of a 1% solution of sulfanilic acid (in 30% acetic acid) were mixed with 5 mL of a 0.1% solution of 1-naphthylamine (in 30% acetic acid). Five drops of this reagent were added to a small portion of gramibactin followed by five drops of concentrated HCl. The resulting red color indicates the presence of nitrite and shows the characteristic reaction for N-nitrosamines.

### ¹⁵N Labeling of Gramibactin

B. graminis was cultured in a 100 mL scale as described above in modified MM9 medium, where (NH₄)₂SO₄ was substituted by (¹⁵NH₄)₂SO₄ (1 mg mL⁻¹). After 24 h the culture supernatant was extracted and purified as described before to yield 2.9 mg of labeled gramibactin (isotope pattern of labeled gramibactin is shown in FIG. 8).

### Synthesis of N²-Benzyloxycarbonyl-N⁶-benzylidene-1-ornithine (2)(6)

KOH (222.2 mg, 3.96 mmol) was dissolved in methanol (4 mL). Cbz-l-Orn (1) (1.005 g, 3.772 mmol) was dissolved in methanol (4 mL), and the methanolic KOH solution was added under stirring at room temperature. Additional 0.1 n methanolic KOH solution (3.3 mL) was titrated into the solution until it became clear. Then, freshly distilled benzaldehyde (405 μL, 4 mmol) was added, followed by molecular sieve 3 Å. The suspension was stirred for 17 h at room temperature. The molecular sieves were filtered off (G3 frit) and washed with methanol. The filtrate was concentrated and lyophilized to give 2 as a white foam (1.175 g; 88%) which was used for the next step without further purification. Obtained NMR data were in accordance with literature data.

### Synthesis of Nitrone 3(6)

Imine 2 (602 mg, 1.53 mmol) was dissolved in methanol (4 mL). Under stirring at 0° C. m-CPBA (635 mg, 3.68 mmol) dissolved in methanol (4 mL) were added slowly. The mixture was stirred for additional two hours at 0° C. The white precipitate was filtered off and washed with ice-cold methanol until the precipitate was dissolved completely. The filtrate was then concentrated and dried at room temperature. The yellowish solid was redissolved in ethyl acetate (10 mL). The pH was adjusted to 1 by adding 1 n HCl. Phases were separated, and the aqueous phase was extracted with ethyl acetate (3×15 mL). The organic phases were combined, washed with sat. NaCl solution and subsequently dried over Na₂SO₄. The solvent was evaporated under reduced pressure, and the slightly yellow solid was dissolved in TFA (2 mL) and CH₂Cl₂ (2 mL) The clear orange solution was stirred at room temperature for one hour. The solvents were removed at ambient temperature and the residue was redissolved in ethyl acetate (10 mL) before adding n-hexane (20 mL) and benzaldehyde (0.3 mL). The mixture was stirred at room temperature for 16 hours and then cooled to 0° C. for 3 hours to complete precipitation. The solid was filtered off, washed with ice-cold ethyl acetate and dried. The crude nitrone 3 was recrystallized from ethyl acetate/n-hexane/iso-propanol (1:1:1) to yield 179.5 mg of 3 (31% over 2 steps). NMR data were in accordance with those reported in the literature. (6)

### Synthesis of N²-benzyloxycarbonyl-l-graminine (4)(6, 7)

Nitrone 3 (140 mg, 0.377 mmol) was dissolved in n-hexane (1.5 mL), 0.5 n HCl (3 mL) and TFA (0.75 mL). The mixture was heated to 60° C. for 15 min. The solvent was evaporated, and the residue was dissolved in CH₂Cl₂ (3 mL) before adding 1 n HCl (4.5 mL). The suspension was heated to 40° C. until it became clear. The solution was then stirred for 1 hour at room temperature. The organic layer was separated, and the aqueous phase was washed with CH₂Cl₂ and n-hexane. It was then concentrated to give 85 mg of a white foam (0.266 mmol, 70%), which was used without further purification. The foam was dissolved in 1 n HCl (260 μL), and a solution of NaNO₂ (18 mg) in 38 μL ddH₂O was added at 0° C. The mixture was slowly warmed to room temperature and then stirred for 30 min. The precipitate was dissolved by adding a few drops of methanol. Compound 4 was purified by preparative HPLC using a Kromasil 100 C₁₈ column (250×20 mm, 5 μm) and gradient elution [solvent A: H₂O+0.1% TFA, solvent B: 83% aqueous MeCN gradient: 10% B over 2 min, 10% B to 100% B over 40 min, flow rate: 20 mL min⁻¹, Rₜ=16.5 min] to give 18.1 mg of 4 as a white powder (0.058 mmol, 22%).

¹H NMR (DMSO-d₆, 500 MHz, 298 K): d 7.65 (d, J=8 Hz, 1H), 7.39-7.28 (m, 5H), 5.03 (s, 2H), 4.06 (t, J=6.8 Hz, 2H), 3.98 (td, J=8.7 and 4.8 Hz, 1H), 1.85 (m, 2H), 1.71 (m, 1H), 1.59 (m, 1H); ¹³C (125 MHz, DMSO-d₆, 298 K) □ 173.5, 156.2, 137.0, 128.4, 127.8, 127.7, 65.5, 61.2, 53.2, 27.6, 23.2.

Incorporation of l-Threonine-2,3-d₂

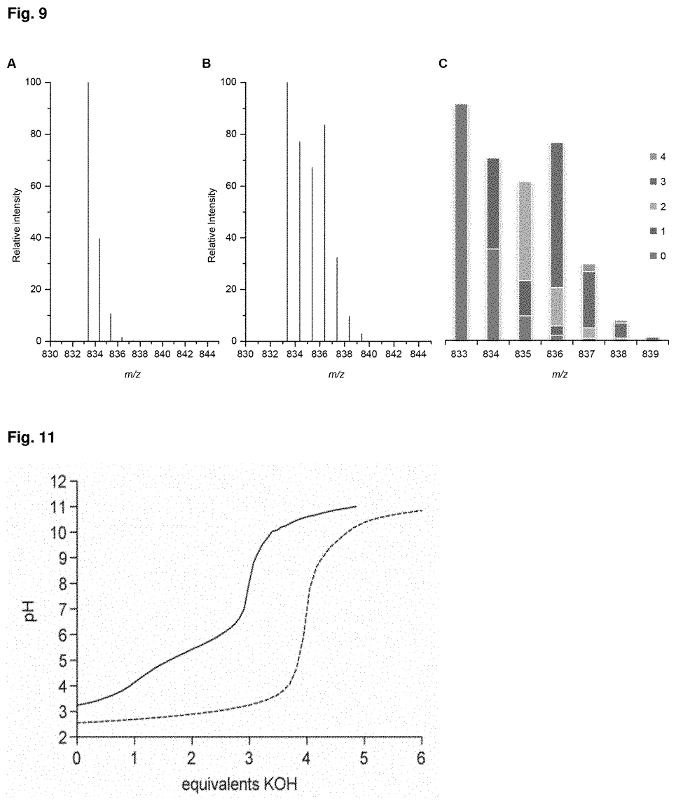

B. graminis was cultured as described above, yet in a modified MM9 medium, where 1-threonine was not included in the amino acid mixture. Three 200-mL cultures were prepared and supplemented with 1-threonine-2,3-d₂ (20 mg). Labeled gramibactin was isolated as described above, yielding 10 mg as a white powder (isotope pattern shown in FIG. 9). For ¹H spectrum, see FIG. S26.

### Amino Acid Analysis of Gramibactin Using Marfey's Method

The absolute configuration of gramibactin was elucidated by Marfey's method (8) using Marfey's reagent (1-fluoro-2,4.dinitrophenyl-5-l-alanine amide, I-FDAA). d,l-Threo-b-hydroxyaspartic acid was obtained from Sigma-Aldrich. The elution order has been shown to be d→l. (9) Cbz-l-graminine (4) was prepared as described above. 0.47 mg gramibactin (0.56 μmol) were hydrolyzed in 7 n HCl_{aq} or 7 n DCI in D20 at 105° C. for 19 h. After lyophilization the sample was resuspended in ddH₂O (50 μL) and 1 m NaHCO₃ (10 μL). Marfey's reagent (10 μL, 10 mg mL⁻¹ in acetone) was added, and the reaction mixture was stirred for 1 h at 40° C. The reaction was quenched by addition of 1 n HCl (10 μL). The reaction mixture was then diluted with ddH₂O (150 μL) and analyzed by analytical HPLC. The reference compounds were derivatized in the same way but were hydrolyzed with HCl only. For analytical HPLC analysis, a Cosmosil 120-5 C₁₈-MS column (250×4.6 mm, Nicalai Tesque) with an elution gradient [solvent A: H₂O+0.1% TFA, solvent B: MeCN, gradient 20% B to 30% B over 30 min, flow rate: 1 mL min⁻¹] was used.

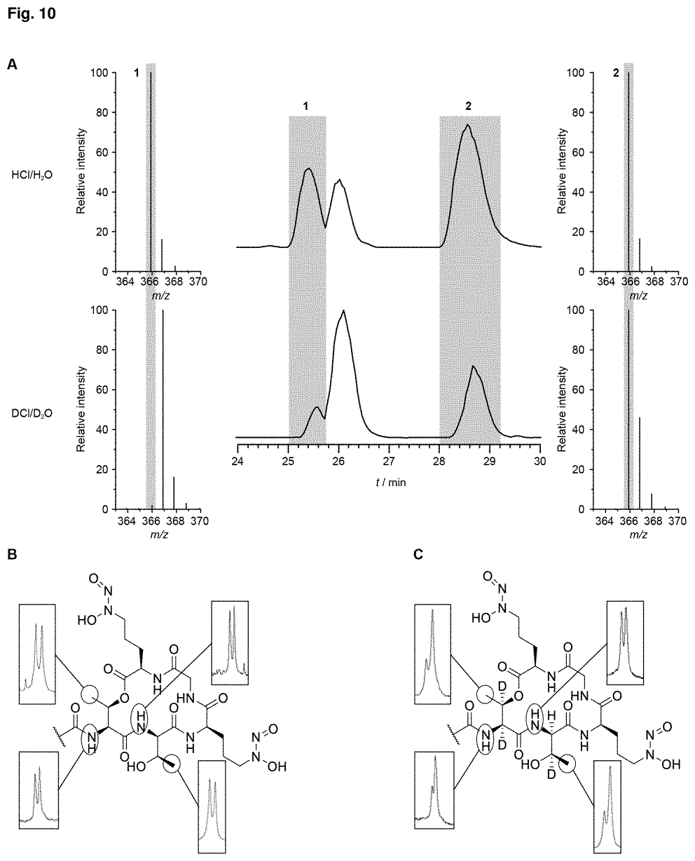

Elucidating the absolute configuration of graminine was not feasible by the typical Marfey approach; hydrolysis of the reference compound and subsequent derivatization with Marfey's reagent led to two different diastereomers, which indicated a facile epimerization during hydrolysis. Using an approach based on MS rather than UV detection we succeeded in assigning the derivatives of the d- and l-enantiomers. Therefore, gramibactin and Cbz-l-graminine were hydrolyzed in DCl/D₂O. When epimerization takes place, the proton in a-position is exchanged by deuterium. The resulting derivatization product would then be one mass unit heavier than of the original one. (10) Thus we were able to assign the absolute configuration of both graminine building blocks in gramibactin as d (FIG. 10A), since experiments with the synthetic reference showed that the derivative of the l-enantiomer elutes earlier.

To elucidate the absolute configurations of the elucidated amino acids, Marfey's method was employed. (8) The configuration of b-hydroxy-aspartate was identified as d-threo and those of the threonine residues as l-threo and d-allo, although their positions remained unclear. Since d-allo threonine would be the epimerization product of 1-threonine, 1-threonine-2,3-d₂ was administered to B. graminis cultures. Deuterium-enriched gramibactin was isolated, and COSY experiments were conducted. While the signal of the amide proton in threonine 2 was unchanged, it was detected as an overlap of a singlet with the normal doublet in threonine 1 (FIG. 7B). The singlet is caused by the presence of deuterium at the a-carbon and a coupling constant that is too low to be properly resolved. In addition, both threonines show a distorted methyl signal in the ¹H spectrum due to deuterium in b-position, indicating that the deuterated threonine is incorporated in both positions. The loss of the deuterium marker in threonine 2 is therefore the product of an epimerization and deuterium-proton exchange. Based on these findings we concluded that threonine 1 has an 1-threo configuration, whereas threonine 2 is d-allo configured. This assignment was confirmed by partial hydrolysis and Marfey's analysis on the isolated gramibactin fragments.

### Preparation of the Gallium(III)-Gramibactin Complex

A solution of gramibactin (5.8 mg) in 800 μL H₂O:ACN:THF (5:2:1) was treated with an excess of Ga(NO₃)₃.xH₂O (4.2 mg). 1 M NaOH (20.9 μL, 3 eq) was added, and the mixture was allowed to stand overnight at room temperature. The gallium complex was purified by preparative HPLC using a Kromasil 100 C₁₈ column (250×20 mm, 5 μm) with an elution gradient [solvent A: H₂O, solvent B: MeCN, gradient: 10% B to 100% B in 15 min, flow rate 18 mL min⁻¹, Rₜ=7.25 min] to yield 1.3 mg of a white powder.

HRESIMS: [M+H+Na]⁺=923.2627 (calculated for C₃₂H₅₁N₁₀O₁₆GaNa 923.2633). For NMR data see Table 7.

### Potentiometric Titrations

Potentiometric ligand competition measurements and calculations have been performed as reported (11) at l=0.1 M in KCl_{(aq)} and at T=25.0±0.1° C., using a Mettler Toledo DL50 apparatus, equipped with a Schott Instruments N6180 combined glass electrode, controlled by LabX light v1.05 software. The titrand solutions consisted in gramibactin (from 0.7 mm to 1 mm), EDTA (as disodium salt) and Fe³⁺ (as FeCl₃) in different gramibactin:Fe³⁺:EDTA ratios, with slight known excess of HCl.

The titration curve of gramibactin (FIG. 11) shows a first inflection point after the addition of one equivalent KOH already at a very low pH, indicating a moiety prone to deprotonation, most likely the free carboxylic group. At the second equivalence point, the curve shows a much flatter slope than at the other two inflection points. This can be explained by a buffering effect of the two N-nitroso-hydroxylamine moieties. The last inflection point marks the complete deprotonation of these two moieties. Analysis of the titration curves for the system gramibactin:Fe³⁺ shows the existence of a considerable drop in pH at very acidic pH, as compared with that of the ligand, thus indicating high affinity of the ligand for this metal ion and measurable complexation already at very low pH. The only visible inflection point in this titration curve appears after the addition of four equivalents KOH. The release of four protons upon metal complexation also fits to our proposed metal binding model.

### Spectrophotometric Titrations

Titrations were carried out at 0.1 m in KCl_{(aq)} and T=25.0±0.1° C. in a glass cuvette (d=10.00 mm) placed in the spectrometer equipped with a thermostated cell holder. The pH was read out by using a inoLab pH 7110 with a combination electrode (SI analytics, ScienceLine Type N 6000 A). The titrant solutions were prepared by addition of defined volumes of individually prepared stock solutions of gramibactin or the respective iron complex (prepared by mixing gramibactin stock solution with different ratios of standardized Fe(NO₃)₃ solution) to the supporting electrolyte (KCl) to obtain the desired ionic strength. In all samples, known slight excess of strong acid (HCl) was added in the titrant solution to lower the starting pH. Measurements were performed by titrating 2.2 mL of the titrant solution with standard KOH_{(aq)} up to pH ˜9 using a Hamilton gas tight syringe. UV/Vis-spectra were recorded after every addition of KOH_{(aq)} followed by magnetic stirring (FIG. 12).

### Plant Material and Growth Conditions

Seeds from maize (Zea mays L. ssp. saccharata) were commercially acquired (Kiepenkerl) and surface sterilized in 4.5% (v/v) sodium hypochlorite for 10 minutes and subsequently rinsed 5 times with 50 mL sterile distilled water. Seeds were germinated in rolled filter papers soaked with sat. CaSO₄ solution for 3 d in at 30° C. in the dark. Seedlings were grown hydroponically in falcon tubes containing 45 mL of a nutrient solution (2 mm Ca(NO₃)₂, 0.75 mm, K₂SO₄, 0.65 mm MgSO₄, 0.5 mm KH₂PO₄, 1 mm KCl, 1 μm H₃BO₃, 1 μm MnSO₄, 0.5 μm ZnSO₄, 1 nm (NH₄)₆Mo₇O₂₄; pH 5.8)(12). For chlorophyll content determination plants were grown in an adjusted nutrient solution that contained 5.25 mm KNO₃, 7.75 mm Ca(NO₃)₂, 4.06 mm MgSO₄, 1 mm KH₂PO₄, 46 μm H₃BO₃, 9.18 μm MnSO₄, 5.4 μm ZnSO₄, 9 μm CuSO₄, 2 μM Na₂MoO₄ (pH adjusted to 5.5 prior to autoclaving)(13). Plants were grown at the lab bench at ambient temperature under a standard fluorescent lamp with 16 h/8 h day/night time.

### In Vitro NO Release Assay

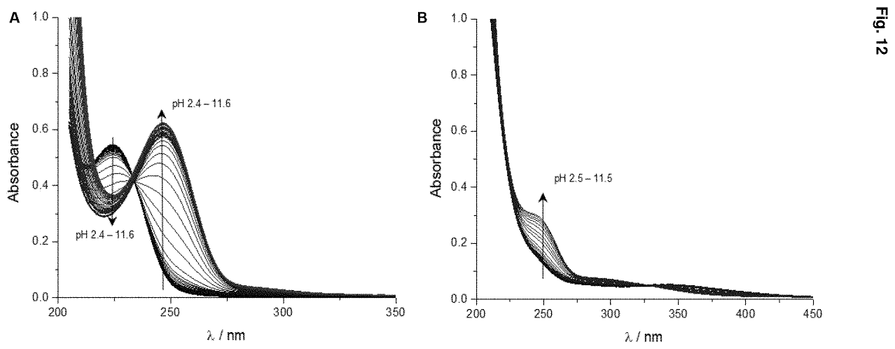

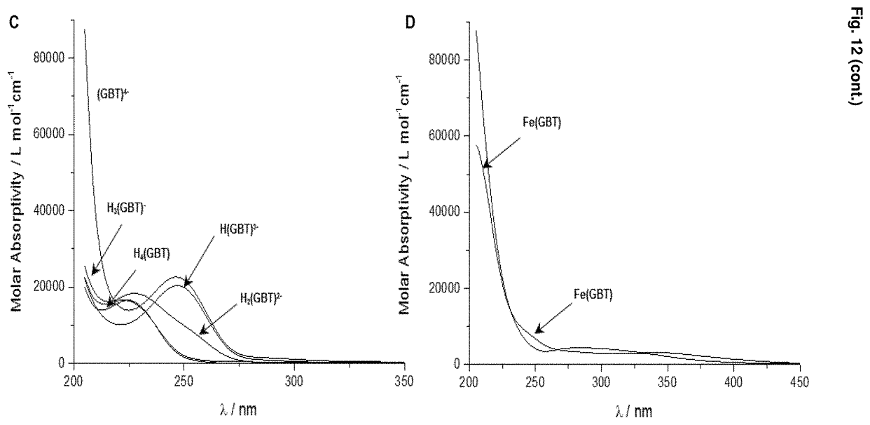

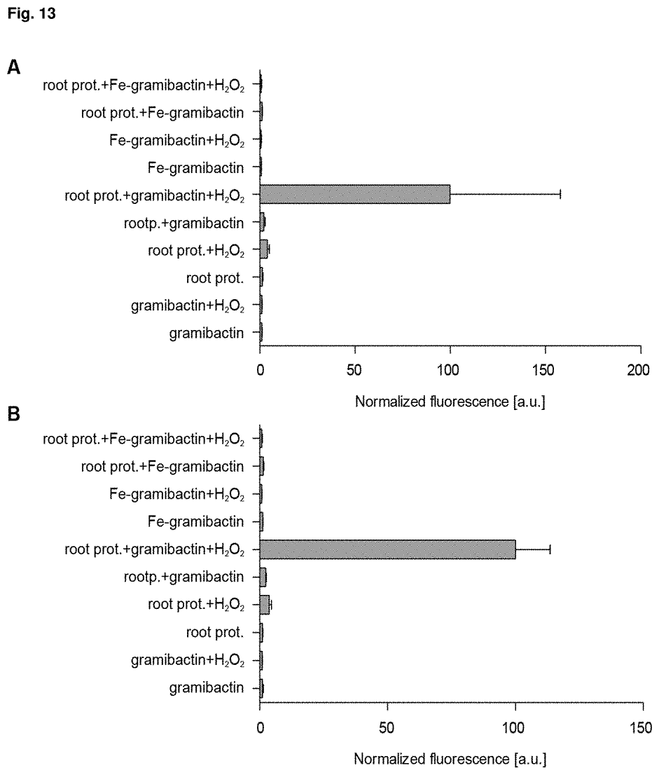

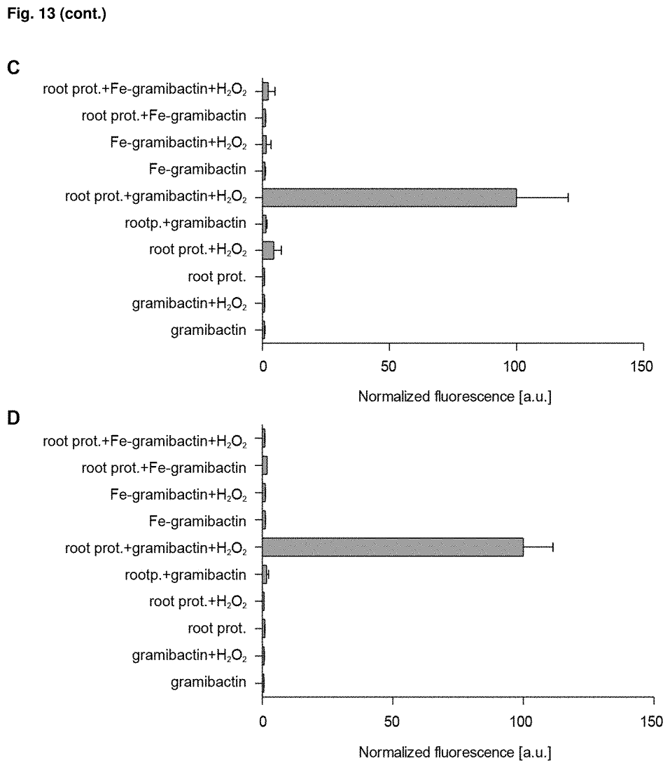

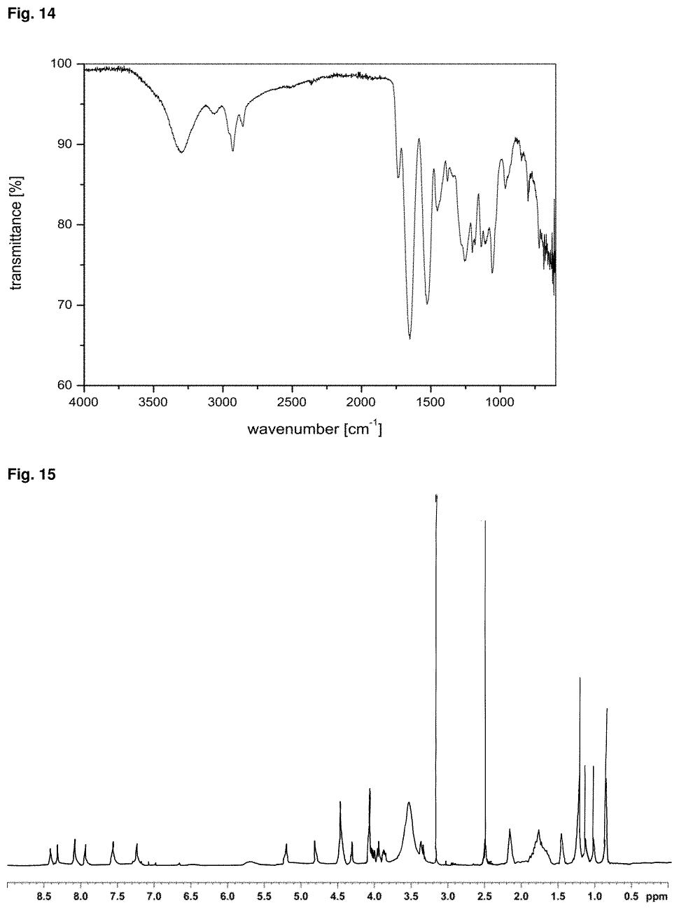

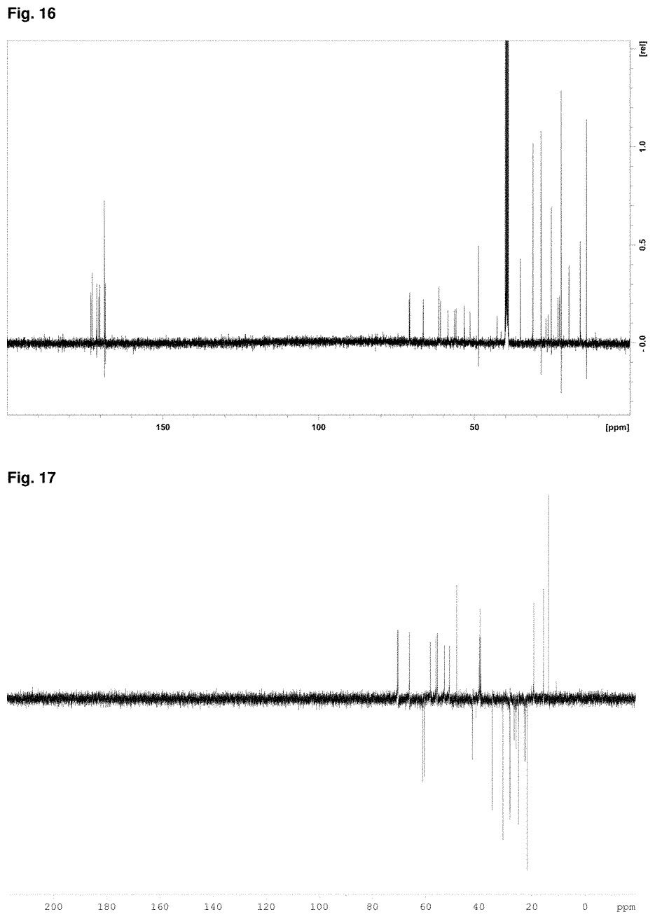

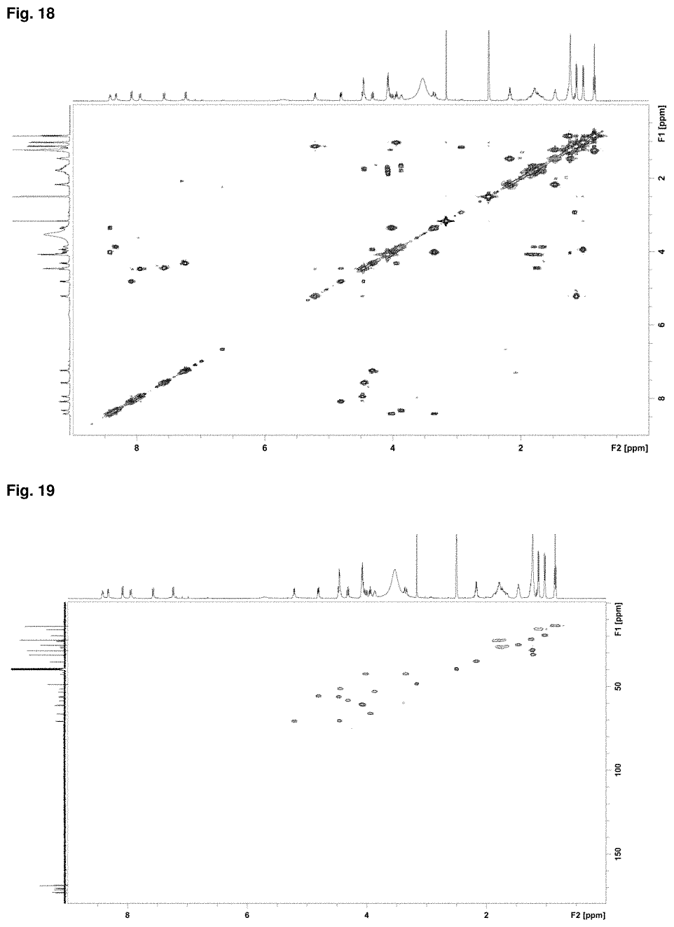

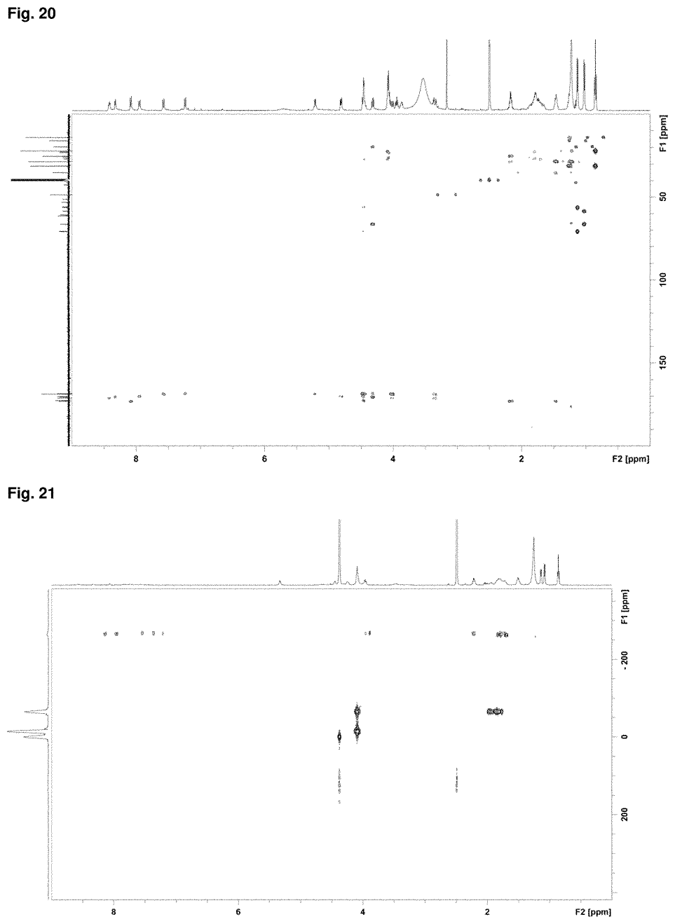

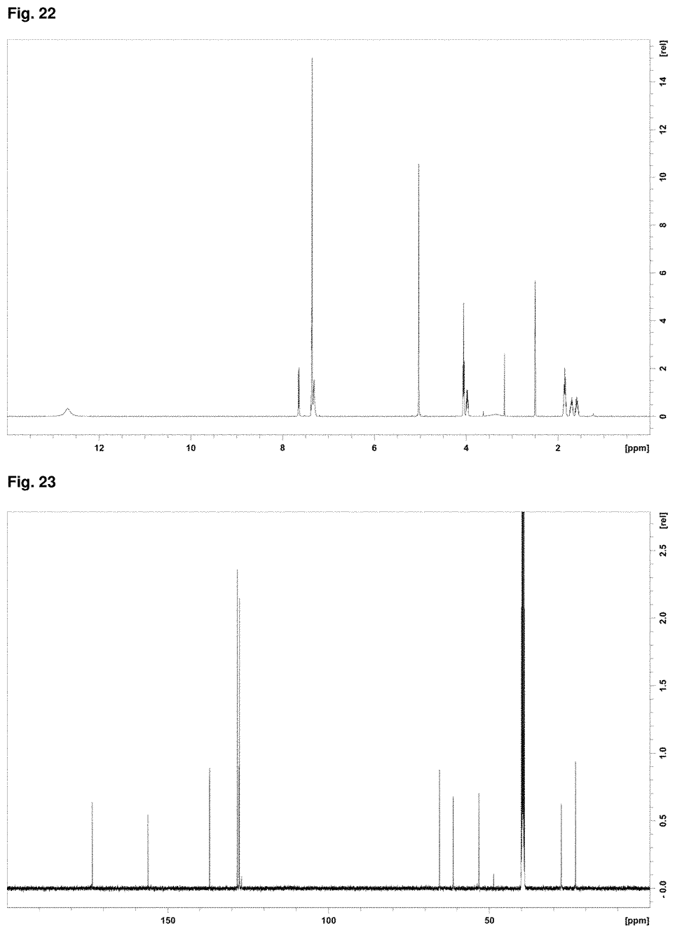

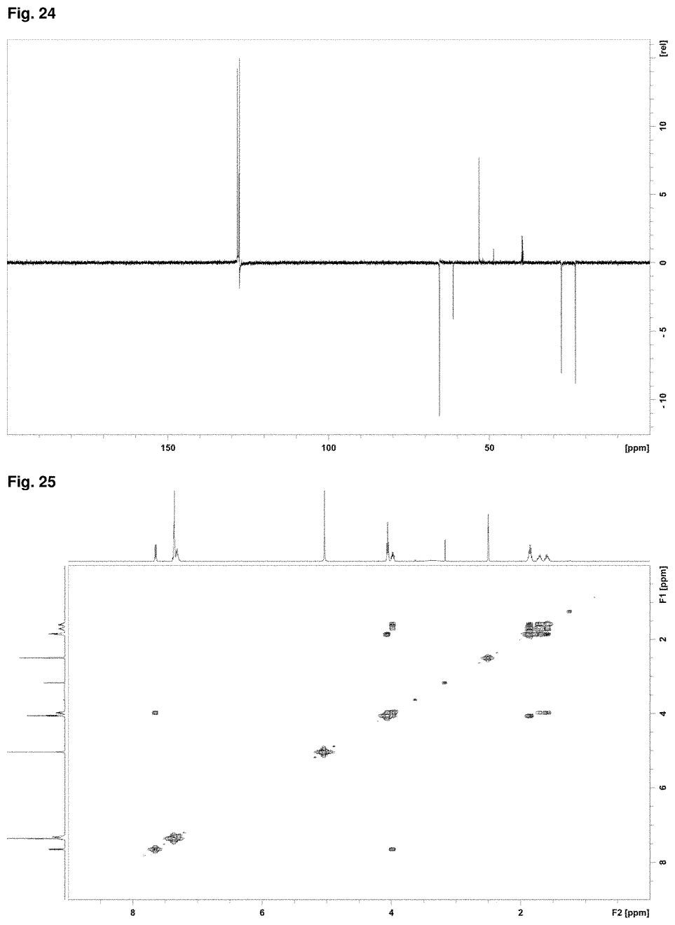

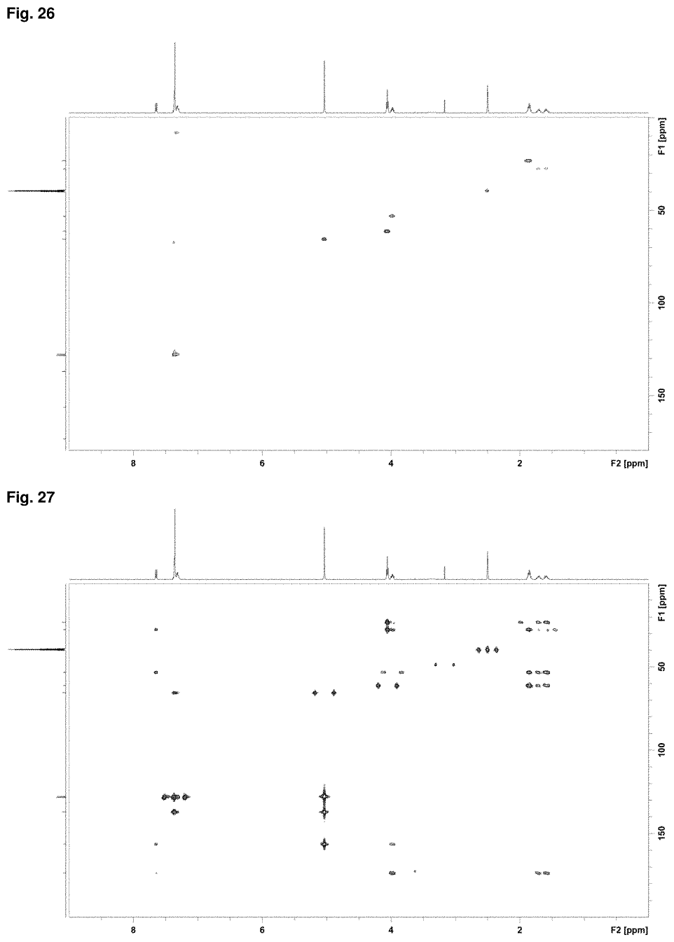

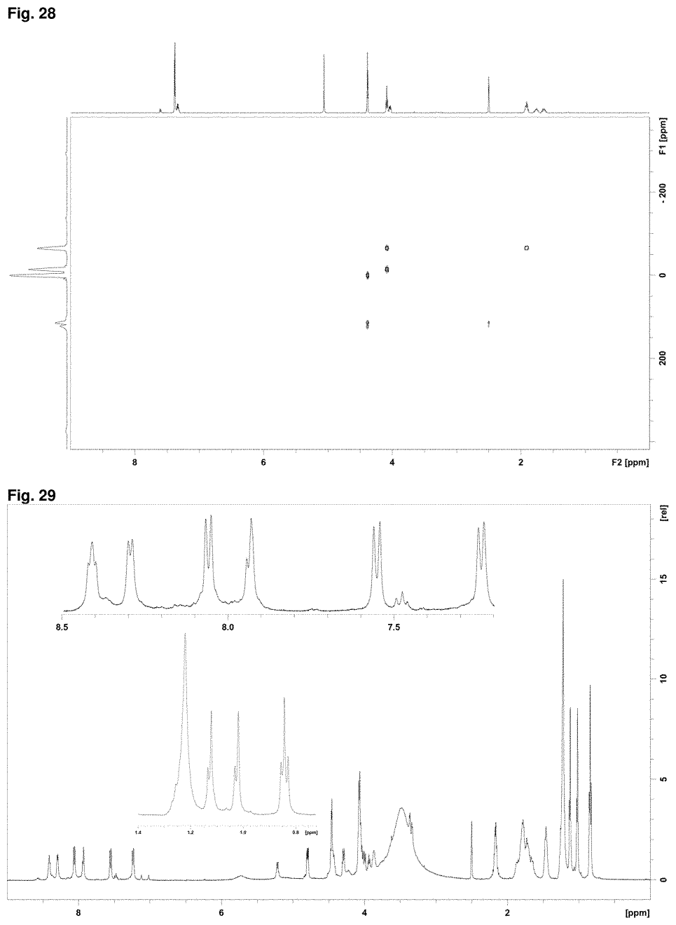

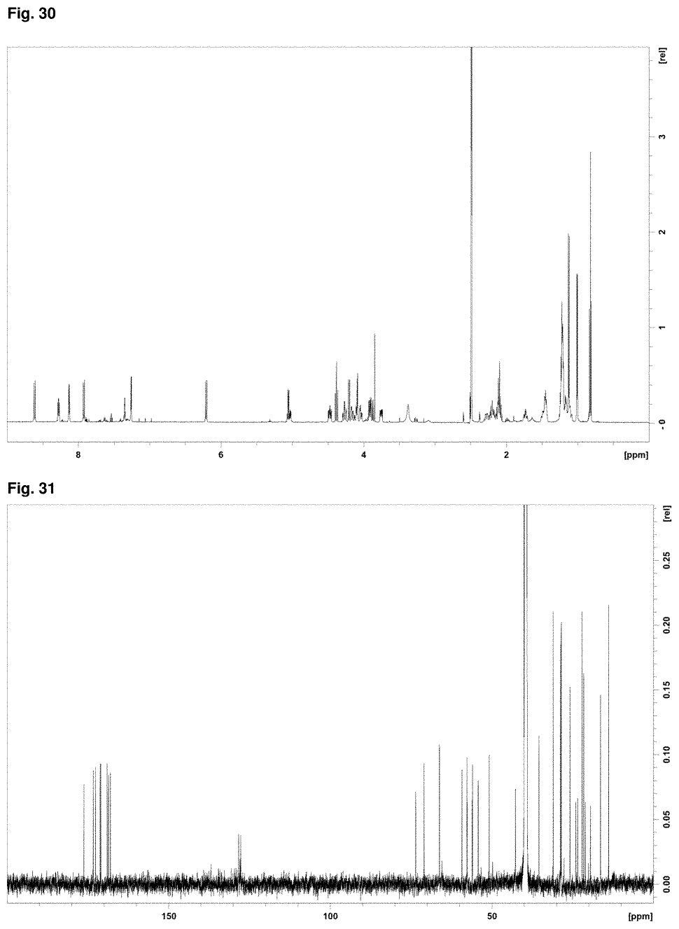

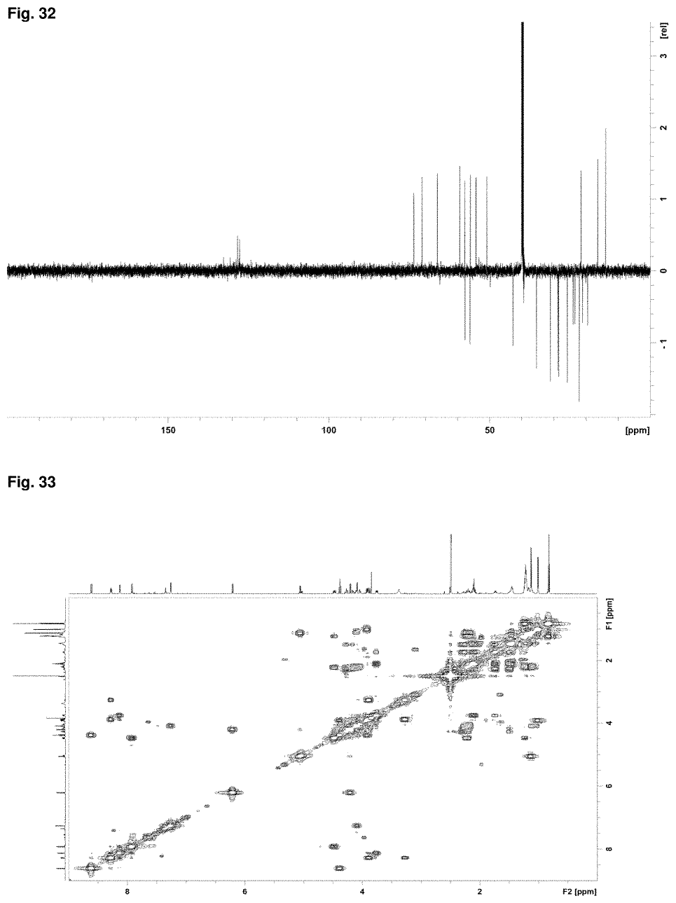

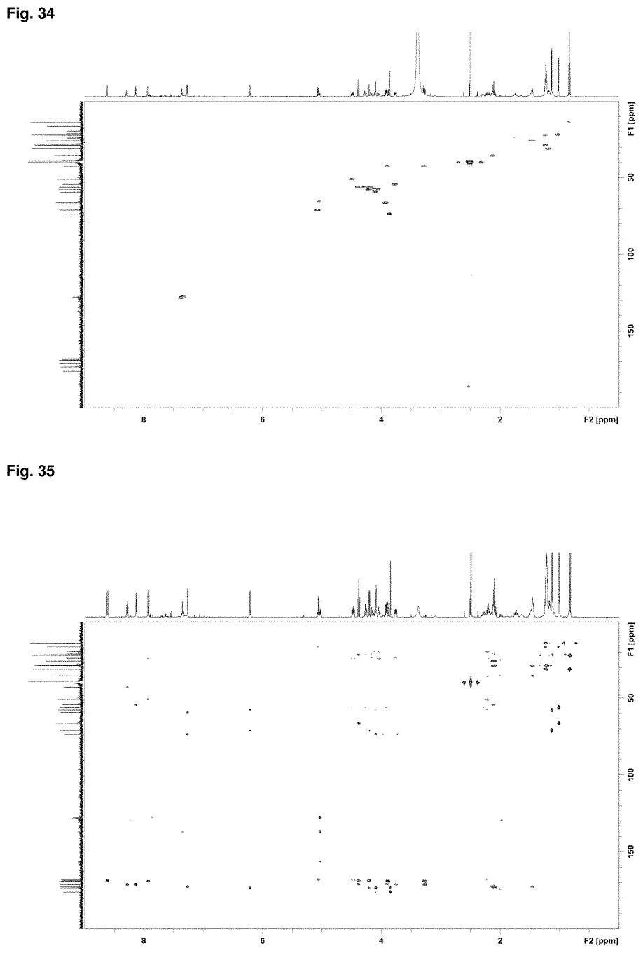

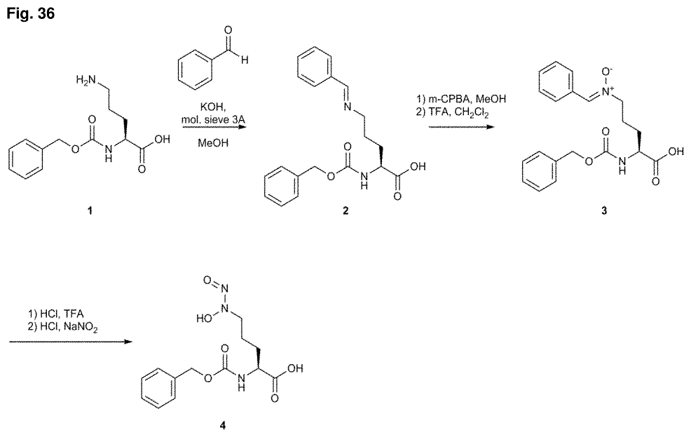

One-week-old plants were harvested and the roots were rinsed with distilled water. After separating the roots from the shoot, the root tissue was frozen using liquid nitrogen and grounded with mortar and pestle. The plant material was then extracted using 1 mL 66 mm NaH₂PO₄ buffer (pH 6). The extract was centrifuged to remove remaining plant material, and 20 μL of the supernatant were used for in vitro assays. 2,3-Diaminonaphthalene (DAN, dissolved in DMF) was used as a probe for released NO. Assays were carried out in 1.5 mL Eppendorf tubes with the following final concentrations: 2 mm gramibactin, 2 mm Fe-gramibactin, 1.2 mm H₂O₂, 0.2 mm DAN. Buffer was added to a final volume of 51.1 μL. After mixing the assays, they were incubated for 20 min at room temperature in the dark. 250 μL 10 mm NaOH was added, and 280 μL of the resulting solution were transferred to a black 96-well plate. Fluorescence was measured using a Varioskan LUX (Thermo Scientific) microplate reader (I_{Ex.}=375 nm, I_{Em.}=415 nm). Fluorescence values were normalized to the sample with the highest fluorescence. Every assay was performed in triplicates and every replicate was measured three times, and the mean of all these replicates was plotted. The assay was done with root extracts from five different plants (FIG. 13).

### In Planta NO Release Assay

Young seedlings (2-3 days old) were placed in a vial containing 4 mL standard nutrient solution (see above) with 10 μm 4,5-diaminofluorescein diacetate and incubated at room temperature for 30 min. Seedlings were then removed, and the root was rinsed with deionized water before placing them in nutrient solution containing 100 μm gramibactin, 100 μm Fe-gramibactin or no additive (control). After 1.5-2 h, roots were placed on styrofoam as a solid support and were cut with a wet razorblade. Upon NO release formed triazolofluorescein was visualized using a Zeiss CLSM 710 confocal laser-scanning microscope (Jena, Germany), and Zen software (Zeiss) has been used to generate the images with I_{Ex}=485 nm and I_{Em}=538 nm. The exact same parameters have been used for all images.

### PET/CT Imaging

⁶⁸Ga was eluted from a ⁶⁸Ge/⁶⁸Ga generator (iThemba Labs, South Africa; initial activity at 22/08/2013: 50 mCi) using a procedure developed by Gebhardt et al. (14) The obtained ⁶⁸Ga solution was buffered with a 1 M KOAc/HOAc buffer to pH 4. If gramibactin was applied, 100 μL of a 1 mg/mL stock solution were added and the solution was incubated for 10 min. Roots of 14-21 d old maize plants were rinsed multiple times with fresh nutrient solution and placed in 35 mL fresh nutrient solution containing either the ⁶⁸Ga eluate or the ⁶⁸Ga-gramibactin complex. Final concentration of Ga in the nutrient solution was 0.11-0.15 pm (˜40-60 MBq, a=1.5·10¹⁸ Bq·g⁻¹) and the used gramibactin concentration 3.24 μM. Plants were incubated for 1 h. In planta imaging was performed with a multimodal Siemens Inveon Small Animal PET/CT system (Siemens Healthcare Medical Imaging). PET acquisitions were conducted with a coincidence-timing window of 3.4 ns and an energy window of 350-650 keV for 1 h. The μCT acquisition protocol used 2,048×3072-pixel axial-transaxial resolution, magnification parameter low (effective pixel size of 108.21 μm), 80 kV at 500 μA, 200 ms exposure time, total rotation of 220° and 120 projections per scan. SUV were calculated using Inveon Research Workplace v4.0 (Siemens Molecular Imaging).

### Chlorophyll Content Determination

Plants were grown hydroponically for 21 days using the above described adjusted nutrient solution. Gramibactin, Fe-gramibactin, EDTA (disodium salt), and Fe-EDTA were dissolved in H₂O:ACN:DMSO (10:2:3) and added to the plants to a final concentration of 50 μm directly after transferring the plants to the nutrient solution. In case of the control plants only the solvent mixture was added. The last developed leaf of each plant (3^{rd} leaf) was removed, and fresh weight was determined. Leaves were then frozen in liquid nitrogen and grounded using mortar and pestle. Acetone was used to extract chlorophylls and the extracts were filtered and lyophylized. The residue was dissolved in 1 mL methanol and 1:10 or 1:20 dilutions with methanol were analysed spectrophotometrically using a Shimadzu UV-1800 spectrophotometer. Samples were measured at 25° C. using discardable plastic cuvettes (d=1 cm). Absorptions at l=666, l=653 nm, and l=470 nm. Chlorophyll contents were calculated using the equations developed by Wellburn. (15)

### Data

Genome sequence information analyzed in this study are available at NCBI with the accession codes NZ_ABLD00000000 (B. graminis C4D1M, https://www.ncbi.nlm.nih.gov/nuccore/NZ_ABLD00000000.1), NZ_ANSK00000000 (B. kururiensis M130, https://www.ncbi.nlm.nih.gov/nuccore/NZ_ANSK00000000.1), NC_015136 (Burkholderia sp. CCGE1001 chromosome 1, https://www.ncbi.nlm.nih.gov/nuccore/NC_015136.1), NC_014539 (Burkholderia sp. CCGE1003 chromosome 1, https://www.ncbi.nlm.nih.gov/nuccore/NC_014539.1), NZ_CP012192 (Burkholderia sp. HB1, https://www.ncbi.nlm.nih.gov/nuccore/NZ_CP012192.1).

### Tables

TABLE 1
Putative functions of deduced proteins encoded in
the gramibactin (grb) biosynthetic gene cluster

| Protein | Size (aa) | Putative function | Sequence (identity/similarity) | Accession No.* |
|---|---|---|---|---|
| GrbA | 207 | Nitroreductase | Nitroreductase (36%/57%) | 3BM1_A |
| | | | Escherichia coli K-12 | |
| GrbB | 77 | MbtH-like protein | MbtH-like protein (70%/81%) | 2PST_X |
| | | | Pseudomonas aeruginosa | |
| | | | PAO1 | |
| GrbC | 292 | Thioesterase II^{†} | Thioesterase (32%/49%) | 3FLB_A |
| | | | Amycolatopsis mediterranei | |
| GrbD | 478 | Unknown function | — | |
| GrbE | 294 | Unknown function | — | |
| GrbF | 548 | ABC Transporter | Pcat1 (26%/42%) | 4RY2_A |
| | | | Ruminiclostridium thermocellum | |
| | | | ATCC 27405 | |
| GrbG | 274 | Transporter | Hmuuv (35%/53%) | 4G1U_C |
| | | | Yersinia pestis | |
| GrbH | 1197 | NRPS (A-T-C) | — | |
| GrbI | 5574 | NRPS (A-T-C-A-T-C-A- | — | |
| | | T-C-A-T-E-C-A-T) | | |
| GrbJ | 1933 | NRPS (C-A-T-E-TE) | — | |
| GrbK | 730 | TonB-dependent | FetA (39%/54%) | 3QLB_A |
| | | transporter | Pseudomonas fluorescens | |
| GrbL | 322 | ABC transporter | FhuD (34%/47%) | 1ESZ_A |
| | | substrate-binding | Escherichia coli | |
| | | protein | | |
| GrbM | 663 | Transporter | Hmuuv (35%/52%) | 4G1U_A |
| | | | Yersinia pestis | |
| GrbN | 718 | TonB-dependent | FetA (26%/42%) | 3QLB_A |
| | | transporter | Pseudomonas fluorescens | |
| GrbO | 423 | Unknown function | — | |

*NCBI or PDB
^{†}GrbC belongs to Family TE18 by ThYme (http://www.enzyme.cbirc.iastate.edu/).(17)

TABLE 2
Prediction of amino acids by AA code of A-domains
in grb NRPS modules (18).

| | | SEQ | | |
|---|---|---|---|---|
| | | ID | | |
| Domain | AA code | NO | Prediction | Resulting AA |
| A₁ | DMITFGCLFK | 4 | Fatty acid | Octanoic acid |
| A₂ | DLTKVGHVGK | 5 | L-Asp | β-Hydroxy-D-Asp |
| A₃ | DFWLIGMVHK | 6 | L-Thr | L-Thr |
| A₄ | DFWNIGMVHK | 7 | L-Thr | D-allo-Thr |
| A₅ | DVHRTGLVAK | 8 | p-Hydroxy- | N^{d}-Nitroso-N^{d}- |
| | | | phenylglycine | hydroxy D-Orn |
| | | | | (Gra) |
| A₆ | DILMIGLIWK | 9 | Gly | Gly |
| A₇ | DVHRTGLVAK | 10 | p-Hydroxy- | N^{d}-Nitroso-N^{d}- |
| | | | phenylglycine | hydroxy D-Orn |
| | | | | (Gra) |

TABLE 3
Primers used in this study.

| | | SEQ | |
|---|---|---|---|
| | Nucleotide sequence | ID | Source or |
| Primer | (5′ to 3′) | NO | reference |
| Bg-NRPS-fw | GCA GGC TGA GGA TTG | 11 | This study |
| | CGG CG | | |
| Bg-NRPS-Pacl | GGT TTA ATT AAC GCC | 12 | This study |
| | CGT TCG ATA AAG CCG C | | |
| Bg-NRPS-Kpnl | GGT GGT ACC TCG CGT | 13 | This study |
| | TTC TCG GCC GTC TC | | |
| Bg-NRPS-Nhel | GGT GCT AGC TCG TGT | 14 | This study |
| | CGA GGT TCA GCG CG | | |
| Bg-NRPS-fw3 | CAT CGG CGG CGC GAG | 15 | This study |
| | TGT CG | | |
| Bg-NRPS-rv3 | CCT CGC TAT CCG TGT | 16 | This study |
| | GCG CG | | |
| Cml-fw-Kpnl | GGT GGT ACC ccc gtc | 17 | This study |
| | agt agc tga aca gg | | |
| Cml-rv-Pacl | GGT TTA ATT AAA ACG | 18 | This study |
| | ACC CTG CCC TGA ACC G | | |

TABLE 4
Plasmids used in this study.

| Plasmid | Relevant characteristics | Source or reference |
|---|---|---|
| pGEM T-easy | TA cloning vector; f1, Amp^{R} | Promega |
| pACYC184 | General cloning vector; p15A, | Invitrogen |
| | Tet^{R}, Cm^{R} | |
| pGEM-Δgrbl | pGEM T-easy containing grbl | This study |
| | insertional Cm^{R} cassette | |

TABLE 5
Bacterial strains used in this study.

| | | |
|---|---|---|
| Strain | Relevant characteristics | Source or reference |

E. coli

| | | |
|---|---|---|
| TOP10 | General cloning host strain | Invitrogen |
| XL1-Blue | General cloning host strain | Stratagene |

B. graminis

| | | |
|---|---|---|
| C4D1M | Wild type; environmental isolate | (19) |
| C4D1M Δgrbl | Cm^{R} cassette inserted into grbl | This study |

TABLE 6
NMR data of gramibactin measured
in DMSO-d₆ (500 MHz, 298K).

| Residue | Position | d_{C} | d_{H} (m, J [Hz]) | |
|---|---|---|---|---|
| HO-Asp | NH | — | 8.08 | (d, 8) |

| CO | 170.2 | (C) | — | |
|---|---|---|---|---|
| Cₐ | 55.8 | (CH) | 4.81 | (dd, 8, 4) |
| C_{b} | 70.7 | (CH) | 4.46 | (m) |

| | | | |
|---|---|---|---|
| COOH | 172.7 | (C) | — |

| | | | | |
|---|---|---|---|---|
| Thr1 | NH | — | 7.95 | (d, 9) |

| CO | 168.4 | (C) | — | |
|---|---|---|---|---|
| Cₐ | 56.3 | (CH) | 4.47 | (m) |
| C_{b} | 70.9 | (CH) | 5.21 | (m) |
| C_{g} | 15.9 | (CH₃) | 1.13 | (d, 6) |

| | | | | |
|---|---|---|---|---|
| Thr2 | NH | — | 7.24 | (d, 8) |

| CO | 170.5 | (C) | — | |
|---|---|---|---|---|
| Cₐ | 58.5 | (CH) | 4.32 | (t, 8) |
| C_{b} | 66.4 | (CH) | 3.94 | (m) |
| C_{g} | 19.6 | (CH₃) | 1.02 | (d, 6) |

| | | | | |
|---|---|---|---|---|
| Gra1 | NH | — | 8.32 | (d, 7) |

| CO | 171.2 | (C) | — | |
|---|---|---|---|---|
| Cₐ | 53.2 | (CH) | 3.87 | (m) |
| C_{b} | 26.2 | (CH₂) | 1.66 | (m) |
| C_{g} | 23.1 | (CH₂) | 1.78 | (m) |
| C_{d} | 60.8 | (CH₂) | 4.07 | (m) |

| | | | | |
|---|---|---|---|---|
| Gly | NH | — | 8.42 | (m) |

| CO | 168.8 | (C) | — | |
|---|---|---|---|---|
| Cₐ | 42.6 | (CH₂) | 3.35 | (dd, 17, 5) & |
| | | | 4.02 | (dd, 17, 7) |

| | | | | |
|---|---|---|---|---|
| Gra2 | NH | — | 7.58 | (d, 9) |

| CO | 168.7 | (C) | — | |
|---|---|---|---|---|
| Cₐ | 51.3 | (CH) | 4.40 | (m) |
| C_{b} | 22.6 | (CH₂) | 1.71 | (m) |
| C_{g} | 26.9 | (CH₂) | 1.76 | (m) |
| C_{d} | 61.3 | (CH₂) | 4.09 | (m) |

| | | | | |
|---|---|---|---|---|
| OA | CO | 173.0 | (C) | — |

| | | | | |
|---|---|---|---|---|
| Cₐ | 35.2 | (CH₂) | 2.17 | (m) |
| C_{b} | 25.3 | (CH₂) | 1.46 | (m) |
| C_{w} | 14.0 | (CH₃) | 0.85 | (t, 7) |

TABLE 7
NMR data of Ga-gramibactin measured
in DMSO-d₆ (600 MHz, 298K).

| Residue | Position | d_{C} | d_{H} (m, J [Hz]) | |
|---|---|---|---|---|
| HO-Asp | NH | — | 7.36 | (d, 2) |

| CO | 173.3 | (C) | — | |
|---|---|---|---|---|
| Cₐ | 59.2 | (CH) | 4.01 | (dd, 5, 1) |
| C_{b} | 73.5 | (CH) | 3.85 | (d, 1) |

| | | | |
|---|---|---|---|
| COOH | 176.2 | (C) | — |

| | | | | |
|---|---|---|---|---|
| Thr1 | NH | — | 6.21 | (d, 10) |

| CO | 168.6 | (C) | — | |
|---|---|---|---|---|
| Cₐ | 57.7 | (CH) | 4.20 | (dd, 10, 2) |
| C_{b} | 71.0 | (CH) | 5.07 | (qd, 6, 2) |
| C_{g} | 16.3 | (CH₃) | 1.13 | (d, 6) |

| | | | | |
|---|---|---|---|---|
| Thr2 | NH | — | 8.62 | (d, 10) |

| CO | 171.0 | (C) | — | |
|---|---|---|---|---|
| Cₐ | 56.0 | (CH) | 4.39 | (t, 10) |
| C_{b} | 66.2 | (CH) | 3.92 | (dq, 10, 6) |
| C_{g} | 21.5 | (CH₃) | 1.02 | (d, 6) |

| | | | | |
|---|---|---|---|---|
| Gra1 | NH | — | 8.14 | (d, 6) |

| CO | 171.2 | (C) | — | |
|---|---|---|---|---|
| Cₐ | 54.2 | (CH) | 3.76 | (m) |
| C_{b} | 23.4 | (CH₂) | 2.12 | (m) & |
| | | | 1.74 | (m) |
| C_{g} | 21.1 | (CH₂) | 1.49 | (m) & |
| | | | 2.28 | (m) |
| C_{d} | 56.1 | (CH₂) | 4.17 | (m) & |
| | | | 4.28 | (td, 14, 3) |

| | | | | |
|---|---|---|---|---|
| Gly | NH | — | 8.28 | (dd, 7, 5) |

| CO | 169.0 | (C) | — | |
|---|---|---|---|---|
| Cₐ | 42.7 | (CH₂) | 3.27 | (dd, 17,5) & |
| | | | 3.89 | (m) |

| | | | | |
|---|---|---|---|---|
| Gra2 | NH | — | 7.93 | (d, 9) |

| CO | 168.0 | (C) | — | |
|---|---|---|---|---|
| Cₐ | 50.8 | (CH) | 4.48 | (m) |
| C_{b} | 24.0 | (CH₂) | 2.22 | (m) & |
| | | | 1.24 | (m) |
| C_{g} | 19.4 | (CH₂) | 2.19 | (m) & |
| | | | 1.11 | (m) |
| C_{d} | 57.6 | (CH₂) | 4.08 | (m) & |
| | | | 4.06 | (m) |

| | | | | |
|---|---|---|---|---|
| OA | CO | 172.6 | (C) | — |

| | | | | |
|---|---|---|---|---|
| C_{w} | 13.8 | (CH₃) | 0.83 | (t, 7) |

### REFERENCES FOR THE ABOVE MATERIALS AND METHODS

1. K. Ishida, T. Lincke, S. Behnken, C. Hertweck, Induced Biosynthesis of Cryptic Polyketide Metabolites in a Burkholderia thailandensis Quorum Sensing Mutant. J. Am. Chem. Soc. 132, 13966-13968 (2010).
2. H. L. Buss, A. Lunge, S. L. Brantley, Etch pit formation on iron silicate surfaces during siderophore-promoted dissolution. Chem. Geol. 240, 326-342 (2007).
3. K. Ishida, T. Lincke, C. Hertweck, Assembly and Absolute Configuration of Short-Lived Polyketides from Burkholderia thailandensis. Angew. Chem. Int. Ed. 51, 5470-5474 (2012).
4. B. C. Louden, D. Haarmann, A. M. Lynne, Use of Blue Agar CAS Assay for Siderophore Detection. Journal of Microbiology and Biology Education: JMBE 12, 51-53 (2011).
5. F. Feigl, C. C. Neto, Spot Tests for Detection of N-Nitroso Compounds (Nitrosamines). Anal. Chem. 28, 1311-1312 (1956).
6. Y.-M. Lin, M. J. Miller, Practical Synthesis of Hydroxamate-Derived Siderophore Components by an Indirect Oxidation Method and Syntheses of a DIG-Siderophore Conjugate and a Biotin-Siderophore Conjugate. J. Org. Chem. 64, 7451-7458 (1999).
7. C. N. Eaton, G. H. Denny, M. A. Ryder, M. G. Ly, R. D. Babson, Improved synthesis of DL-alanosine. J. Med. Chem. 16, 289-290 (1973).
8. R. Bhushan, H. Bruckner, Marfey's reagent for chiral amino acid analysis: a review. Amino Acids 27, 231-247 (2004).
9. K. Fujii, Y. Ikai, T. Mayumi, H. Oka, M. Suzuki, K.-i. Harada, A Nonempirical Method Using LC/MS for Determination of the Absolute Configuration of Constituent Amino Acids in a Peptide: Elucidation of Limitations of Marfey's Method and of Its Separation Mechanism. Anal. Chem. 69, 3346-3352 (1997).
10. D. R. Goodlett, P. A. Abuaf, P. A. Savage, K. A. Kowalski, T. K. Mukherjee, J. W. Tolan, N. Corkum, G. Goldstein, J. B. Crowther, Peptide chiral purity determination: hydrolysis in deuterated acid, derivatization with Marfey's reagent and analysis using high-performance liquid chromatography-electrospray ionization-mass spectrometry. J. Chromatogr. A 707, 233-244 (1995).
11. C. Bretti, R. M. Cigala, G. Lando, D. Milea, S. Sammartano, Sequestering Ability of Phytate toward Biologically and Environmentally Relevant Trivalent Metal Cations. J. Agric. Food Chem. 60, 8075-8082 (2012).
12. E. Bar-Ness, Y. Hadar, Y. Chen, V. Romheld, H. Marschner, Short-Term Effects of Rhizosphere Microorganisms on Fe Uptake from Microbial Siderophores by Maize and Oat. Plant Physiol. 100, 451-456 (1992).
13. M. Graziano, M. V. Beligni, L. Lamattina, Nitric Oxide Improves Internal Iron Availability in Plants. Plant Physiol. 130, 1852-1859 (2002).
14. P. Gebhardt, T. Opfermann, H. P. Saluz, Computer controlled 68Ga milking and concentration system. Appl. Radiat. Isot. 68, 1057-1059 (2010).
15. A. R. Wellburn, The Spectral Determination of Chlorophylls a and b, as well as Total Carotenoids, Using Various Solvents with Spectrophotometers of Different Resolution. J. Plant Physiol. 144, 307-313 (1994).
16. T. Stachelhaus, H. D. Mootz, M. A. Marahiel, The specificity-conferring code of adenylation domains in nonribosomal peptide synthetases. Chem. Biol. 6, 493-505 (1999).
17. D. C. Cantu, Y. Chen, M. L. Lemons, P. J. Reilly, ThYme: a database for thioester-active enzymes. Nucleic Acids Res. 39, D342-D346 (2011).
18. M. Rottig, M. H. Medema, K. Blin, T. Weber, C. Rausch, O. Kohlbacher, NRPSpredictor2—a web server for predicting NRPS adenylation domain specificity. Nucleic Acids Res. 39, W362-367 (2011).
19. V. Viallard, I. Poirier, B. Cournoyer, J. Haurat, S. Wiebkin, K. Ophel-Keller, J. Balandreau, Burkholderia graminis sp. nov., a rhizospheric Burkholderia species, and reassessment of [Pseudomonas] phenazinium, [Pseudomonas] pyrrocinia and [Pseudomonas] glathei as Burkholderia. Int. J. Syst. Evol. Microbiol. 48, 549-563 (1998).

1. An isolated peptide siderophore, comprising one or more N-nitroso-hydroxylamine ligands.

2. The peptide siderophore of claim 1, comprising two side chains, each with a N-nitroso-hydroxylamine ligand, and a further side chain.

3. The peptide siderophore of claim 1, comprising a peptide of X1-X2-X3-Gra1-X4-Gra2, wherein:
X1 to X4 are amino acids and wherein at least one of X1 to X4 comprises a side chain with at least two atoms capable of forming one or more Fe-chelating ligands; and
Gra1 and Gra2 may be the same or different and are amino acids comprising a side chain with an N-nitroso-hydroxylamine ligand.

4. The peptide siderophore of claim 1, comprising a peptide of Asp-Thr1-Thr2-Gra1-Gly-Gra2, wherein Asp comprises a side chain with an additional hydroxy group adjacent to the carboxylic acid group.

5. The peptide siderophore of claim 1, wherein the peptide is cyclic.

6. The peptide siderophore of claim 4, wherein the side chain of Thr1 and the C-terminus of Gra2 are linked to form a cyclic peptide.

7. The peptide siderophore of claim 5, comprising X2-X3-Gra1-X4-Gra2, wherein:
X2 to X4 are amino acids and wherein at least one of X2 and/or X3 comprises a side chain with at least two atoms capable of forming one or more Fe-chelating ligands; and
the side chain of X2 and the C-terminus of Gra2, or the N-terminus of X2 and the C-terminus of Gra2, are linked to form a cyclic peptide.

8. The peptide siderophore of claim 1, wherein the N-nitroso-hydroxylamine ligand is present in an amino acid of the structure: 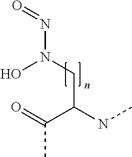
wherein n is a value from 1 to 7.

9. The peptide siderophore of claim 1, wherein the N-nitroso-hydroxylamine ligand is present in an amino acid of the structure:

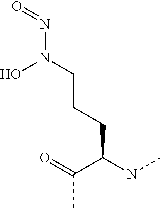

10. The peptide siderophore of claim 1, in the form of a cyclic peptide siderophore according to Formula I: 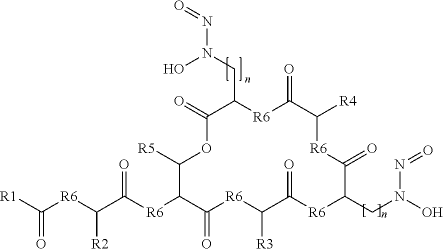
wherein:
R1 is C1-C12 alkyl, alkoxy, carboxyl or hydroxy carboxyl, or one or more amino acids, or the remaining part of an amino acid (—NH—CR—) additional to the C═O group to which R1 is attached, wherein R is the amino acid side chain);
R2 to R5 may be the same or different, and are selected from the group consisting of a side chain of an amino acid, H, OH, C1-C7 alkyl, alkoxy, carboxyl and hydroxy carboxyl;
R6 can be the same or different, wherein R6 is O or NH, and wherein at least one of R6 is NH;
n is a value from 1 to 7 wherein n for different substituents may be the same or different;
and wherein R1 to R5 comprise between them at least two atoms capable of forming one or more Fe-chelating ligands.

11. The cyclic peptide siderophore of claim 10, according to Formula II: 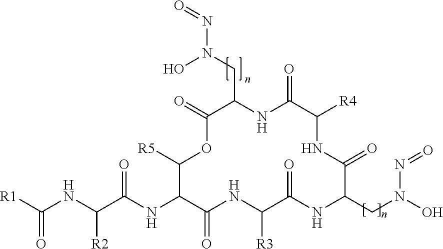
wherein:
R1 is C1-C12 alkyl, alkoxy, carboxyl or hydroxy carboxyl, or one or more amino acids, or the remaining part of an amino acid (—NH—CR—) additional to the C═O group to which R1 is attached, wherein R is the amino acid side chain;
R2 to R5 may be the same or different, and are selected from the group consisting of a side chain of an amino acid, H, OH, C1-C7 alkyl, alkoxy, carboxyl and hydroxy carboxyl;
n is a value from 1 to 7;
and wherein R1 to R5 comprise between them at least two atoms capable of forming one or more Fe-chelating ligands.

12. The cyclic peptide siderophore of claim 11, wherein
R1 is C1-C12 alkyl, alkoxy, carboxyl or hydroxy carboxyl;
R2 is the side chain of Asp or Glu, optionally comprising an additional hydroxy group adjacent, alpha or beta, to the carboxylic acid group;
R3 is the side chain of Ser, Thr, Asn or Gln;
R4 is H or C1-C7 alkyl;
R5 is H or C1-C7 alkyl;
n is a value from 2 to 5.

13. The cyclic peptide siderophore of claim 11, having the following structure:

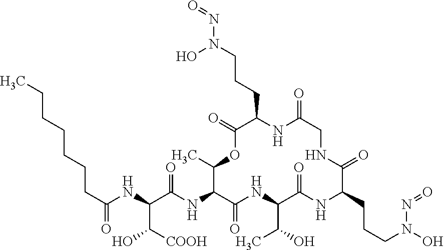 [SMILES: CCCCCCCC(=O)N[C@@H](C(=O)N[C@@H]1C(=O)N[C@H]([C@@H](C)O)C(=O)N[C@H](CCCN(O)N=O)C(=O)NCC(=O)N[C@H](CCCN(O)N=O)C(=O)O[C@@H]1C)[C@@H](O)C(=O)O]

14. A bacterial cell that produces and/or secretes the peptide siderophore of claim 1.

15. A composition comprising the peptide siderophore of claim 1 and/or a bacterial cell producing and/or secreting a peptide siderophore of claim 15.

16. The composition of claim 15, comprising the peptide siderophore in its corresponding iron-complex.

17. A method for promoting plant growth comprising administering the peptide siderophore of claim 1, or a composition comprising the peptide siderophore of claim 1, or a bacterial cell expressing a peptide siderophore according to claim 1, in an amount effective to promote growth of said plant.

18. The method for promoting plant growth of claim 17, wherein the method comprises administering a bacterial cell that produces and/or secretes the peptide siderophore of claim 1, wherein the bacterial cell expressing a peptide siderophore comprises a nonribosomal peptide synthetase (NRPS) gene cluster (grb), comprising associated genes encoding a TonB-dependent siderophore receptor and an iron-hydroxamate transporter ATP binding protein.

19. The method for promoting plant growth according to claim 18, wherein the bacterial cell is selected from a Burkholderia species.

20. A method for promoting root growth or development, improving stress tolerance and/or for increasing crop yields of a plant, comprising administering a peptide siderophore of claim 1 or a composition comprising the peptide siderophore of claim 1, or a bacterial cell that produces and/or secretes the peptide siderophore of claim 1, in an amount effective to promote root growth, stress tolerance and/or for increasing crop yields of said plant.

21. A method for delivering nitric oxide (NO) to a plant, comprising administering a peptide siderophore of claim 1 or a composition comprising the peptide siderophore of claim 1, or a bacterial cell that produces and/or secretes the peptide siderophore of claim 1, in an amount effective to deliver NO to said plant.

22. A method for enhancing chlorophyll production in a plant, comprising administering a peptide siderophore of claim 1 or a composition comprising the peptide siderophore of claim 1, or a bacterial cell that produces and/or secretes the peptide siderophore of claim 1, in an amount effective to enhance chlorophyll production in said plant.

23. (canceled)

24. The peptide siderophore of claim 5, wherein the peptide comprises a cyclic structure of 2 to 10 amino acids.

25. The peptide siderophore of claim 2, wherein the further side chain has an N-nitroso-hydroxylamine ligand, or one or more hydroxy carboxylic acid, hydroxamate, catecholate and/or salicylate ligands.
